(19) United States
(12) Patent Application Publication
(10) Pub. No.: US 2021/0236619 A1
(43) Pub. Date: Aug. 5, 2021

(54) STEM CELLS COMPRISING SYNTHETIC CHIMERIC VACCINIA VIRUS AND METHODS OF USING THEM

(71) Applicant: TONIX PHARMA HOLDINGS LIMITED, HAMILTON (BM)

(72) Inventor: Seth Lederman, South Dartmouth, MA (US)

(21) Appl. No.: 17/049,741

(22) PCT Filed: May 2, 2019
(86) PCT No.: PCT/US2019/030488

§ 371 Date: Oct. 22, 2020

Related U.S. Application Data
(60) Provisional application No. 62/666,013, filed on May 2, 2018.

(51) Int. Cl. A61K 39/12; C12N 7/00; A61P 35/00; A61K 35/28

(52) U.S. Cl. CPC A61K 39/12; C12N 7/00; A61K 2039/5252; A61K 35/28; A61P 35/00

(57) ABSTRACT

The invention relates in various aspects to stem cells comprising a synthetic chimeric poxvirus (scPV), which can be used for the treatment of cancer or other infectious diseases. It also relates to methods for delivering the scPV comprising infecting the stem cells with the scPV and administering the infected stem cells to a subject.

### BACKGROUND OF THE DISCLOSURE

A Sequence Listing associated with this application is being submitted electronically via EFS-Web in text format and is hereby incorporated by reference in its entirety into the specification. The name of the text file containing the Sequence Listing is 104545-0032-WO1-SequenceListing.txt. The text file, created on May 2, 2019, is 882,663 bytes in size.

Poxviruses (members of the Poxviridae family) are double-stranded DNA viruses that can infect both humans and animals. Poxviruses are divided into two subfamilies based on host range. The Chordopoxviridae subfamily, which infects vertebrate hosts, consists of eight genera, of which four genera (Orthopoxvirus, Parapoxvirus, Molluscipoxvirus, and Yatapoxvirus) are known to infect humans. Smallpox is caused by infection with variola virus (VARV), a member of the genus Orthopoxvirus (OPV). The OPV genus comprises a number of genetically related and morphologically identical viruses, including camelpox virus (CMLV), cowpox virus (CPXV), ectromelia virus (ECTV, “mousepox agent”), horsepox virus (HPXV), monkeypox virus (MPXV), rabbitpox virus (RPXV), raccoonpox virus, skunkpox virus, Taterapox virus, Uasin Gishu disease virus, vaccinia virus (VACV), variola virus (VARV) and volepox virus (VPV). Other than VARV, at least three other OPVs, including VACV, MPXV and CPXV, are known to infect humans.

In the ongoing search for therapeutics that are capable of eliminating or reducing tumor cells, oncolytic viruses have shown great potential in preclinical studies. These viruses are typically genetically engineered or have a natural tropism for tumor cells, so as to only replicate in and kill neoplastic cells. Among therapeutic viruses, oncolytic Vaccinia virus is one of the most promising candidates for cancer therapy. As an example, JX-594 is a Vaccinia virus with TK gene deletion and GM-CSF (a cytokine able to stimulate the immune system to kill tumor cells) gene insertion and is currently undergoing phase III trials (Park S H et al. Phase 1b trial of biweekly intravenous Pexa-Vec (JX-594), an oncolytic and immunotherapeutic vaccinia virus in colorectal cancer. Mol Ther (2015); 23(9):1532-40).

To improve delivery of viral therapeutics and circumvent antiviral immunity, a number of studies have explored the possibility of using infected cells as delivery vehicles for oncolytic viruses (Garcia-Castro, J., et al., Treatment of metastatic neuroblastoma with systemic oncolytic virotherapy delivered by autologous mesenchymal stem cells: an exploratory study. Cancer Gene Ther, 2010. 17(7): 476-83; Coukos, G., et al., Use of carrier cells to deliver a replication-selective herpes simplex virus-1 mutant for the intraperitoneal therapy of epithelial ovarian cancer. Clin Cancer Res, 1999. 5(6): 1523-37; Komarova, S., et al., Mesenchymal progenitor cells as cellular vehicles for delivery of oncolytic adenoviruses. Mol Cancer Ther, 2006. 5(3): 755-66). Mesenchymal stem cells have shown great promise in this respect.

Although promising, these studies have been limited by their inability to explore the therapeutic efficacy of mesenchymal stem cells loaded with oncolytic viruses. Additionally, cell therapies, such as administration of stem cells, have been associated with formation of malignant cancers in subjects receiving stem cell therapies. While the potential of cellular therapy in the treatment of diseases, disorders and injury is significant, the formation of tumors, such as teratomas, as a result of such treatment is an unacceptable outcome.

Accordingly, there exists a need for methods of delivering oncolytic viruses and safely administering cellular compositions that reduce the risk of tumor formation in subjects receiving cellular therapy.

### SUMMARY OF THE DISCLOSURE

The present invention, in one aspect, provides stem cells comprising a synthetic chimeric poxvirus (scPV), which can be used for the treatment of cancer or other infectious diseases. Because chemical genome synthesis is not dependent on a natural template, a plethora of structural and functional modifications of the viral genome are possible. Chemical genome synthesis is particularly useful when a natural template is not available for genetic replication or modification by conventional molecular biology methods.

Therefore, in one aspect, the invention relates to an isolated stem cell or population thereof comprising a synthetic chimeric poxvirus (scPV), wherein the virus is replicated and reactivated from DNA derived from synthetic DNA, the viral genome of said virus differing from a wild type genome of said virus in that it is characterized by one or more modifications.

In another aspect, the invention relates to a pharmaceutical composition comprising the isolated stem cells of the invention, and a pharmaceutically acceptable carrier.

In another aspect, the invention relates to a method for delivering the scPV of the invention to a subject, comprising infecting the stem cells of the invention and administering the scPV-infected stem cells into the subject.

In another aspect, the invention relates to a method of treating or preventing cancer in a subject, comprising administering the stem cells of the invention or the pharmaceutical composition of the invention to the subject, to thereby contact the cancer cells of the subject with the scPV.

In another aspect, the invention relates to a method of treating a variola virus infection, comprising administering to a subject in need thereof the stem cells of the invention or the pharmaceutical composition of the invention.

### BRIEF DESCRIPTION OF THE DRAWINGS

This patent application contains at least one drawing executed in color. Copies of this patent application with color drawings will be provided by the Office upon request and payment of the necessary fee.

The foregoing summary, as well as the following detailed description of the disclosure, will be better understood when read in conjunction with the appended drawings. For the purpose of illustrating various aspects of the invention there are shown in the drawings embodiment(s) which are presently preferred. It should be understood, however, that the invention is not limited to the precise arrangements and instrumentalities shown.

FIGS. 1A and 1B. Schematic representation of the linear dsDNA HPXV genome (strain MNR; Genbank Accession DQ792504). A. FIG. 1A illustrates the unmodified genome sequence of HPXV genome with individual HPXV genes (purple) and the naturally occurring AarI and BsaI sites indicated. B. FIG. 1B depicts the modified synthetic chimeric HPXV (scHPXV) genome that was chemically synthesized using the overlapping genomic DNA fragments (shown in red). The engineered SapI restriction sites that were used to ligate the VACV terminal hairpin loops onto the ITRs, along with the unmodified BsaI sites in the left and right ITR fragments, are also shown. The SapI sites were located in plasmid vector sites immediately to the left and right ends of the Left Inverted Terminal Repeat (LITR) and Right Inverted Terminal Repeat (RITR), respectively.

FIG. 2A-2C. Detailed schematic representation of the modified scHPXV YFP-gpt::095 genome and VACV (WR strain) terminal hairpin loops. A. FIG. 2A depicts the modified scHPXV YFP-gpt::095 genome. The unmodified BsaI sites are shown as blue lines on the genome. The novel AvaI and StuI restriction sites that were created in HPXV044 (the VACV F4L homolog) are also marked (green lines). The location of the selectable marker yellow fluorescent protein/guanosine phosphoribosyl transferase (yfp/gpt) in the HPXV095 locus (the VACV J2R homolog) of Frag_3 is also shown (yellow). B. FIG. 2B depicts the nucleotide sequence of the S (SEQ ID NO: 11) and F (SEQ ID NO: 12) forms of the terminal hairpin loop, and the color coding is explained in (C). C. FIG. 2C depicts the secondary structure predictions of the F and S forms of terminal hairpin loops (SEQ ID NOS 12 and 11, respectively) that are covalently attached to the terminal ends of the linear dsDNA genomes of VACV. The terminal loop sequence is highlighted in green. The concatamer resolution sequence is boxed in red.

FIG. 3. The ˜70 bp VACV terminal hairpin was ligated to the left and right HPXV ITR fragments. FIG. 3 depicts agarose gel electrophoresis of the left and right ITR fragments following ligation of the ˜70 bp terminal hairpin to the 1472 bp ITR fragment cut with SapI. The ligated DNAs were subsequently cut with PvuII to facilitate detection of the small change in size caused by the addition of the hairpins.

FIG. 4A-4B. PCR analysis and restriction digestion of scHPXV YFP-gpt::095 genomes confirm successful reactivation of scHPXV YFP-gpt::095. A. FIG. 4A depicts pulse field gel electrophoresis (PFGE) of VACV-WR and scHPXV YFP-gpt::095 genomic DNAs. Virus DNAs was digested with BsaI, HindIII, or left untreated, and were then separated on a 1% Seakem gold agarose gel for 14 h at 14° C. at 5.7V/cm with a switch time of 1 to 10 seconds. A slight difference in size between the intact VACV and scHPXV YFP-gpt::095 genomes was observed. The faint bands marked with an asterisk (*) are either incomplete DNA digestion products or could be cut mitochondrial DNA fragments that often contaminate VACV virion preparations. B. FIG. 4B depicts conventional agarose gel electrophoresis of VACV-WR and scHPXV YFP-gpt::095 genomic DNA digested with BsaI or HindIII. DNA fragments were visualized by staining gels with SybrGold DNA stain.

FIG. 5A-5C. The BsaI sites in scHPXV YFP-gpt::095 region 96,050 to 96,500 were correctly mutated. FIG. 5A depicts Illumina sequence reads that mapped to one region of the HPXV (DQ792504) genome. Only a small fraction of the reads is shown. The conflicts in the sequencing reads at pos. 96,239 and pos. 96437 are highlighted in blue and yellow, respectively. FIG. 5B depicts the magnification of the Illumina sequencing reads from scHPXV YFP-gpt::095 that mapped to HPXV (DQ792504) near pos. 96239. A nucleotide substitution (T96239C) was detected (refer to Table 2). Figure discloses SEQ ID NOS 68-70, 69, 71, 72, 69, 73-76, 69, 77-79, 69, 69, 80, 81, 69, 82, 69, 83, 69, 84, 69, 84, 85, 69, 69, 86, 69, 69, 87, 69, 69, 69, 69, 88, 69, 69, 69, 89, and 90, respectively, in order of appearance. FIG. 5C depicts the magnification of the Illumina sequencing reads from scHPXV YFP-gpt::095 that mapped to HPXV (DQ792504) at pos. 96437. A nucleotide substitution (A96437C) was detected (refer to Table 2). These mutations were introduced into the clones used to assemble scHPXV YFP-gpt::095 so as to delete undesirable BsaI recognition sites (GGTCTC). Figure discloses SEQ ID NOS 91-94, 92, 95, 92, 92, 92, 96, 92, 97, 98, 92, 92, 92, 92, 92, 99-101, 92, 102, 103, 92, 104-109, 92, 110, 92, 92, 111, 103, 112, 92, 113, 114, 92, 92, 115, and 116, respectively, in order of appearance.

FIG. 6A-6C. ScHPXV YFP-gpt::095 grows like other Orthopoxviruses but exhibits a small plaque phenotype in BSC-40 cells. A. FIG. 6A illustrates the multi-step growth of VACV-WR, DPP15, CPXV, and scHPXV YFP-gpt::095 in BSC-40 (top left panel), HeLa (top middle panel), primary HEL (top right panel), and Vero (bottom left panel) cell lines. B. FIG. 6B illustrates plaque size comparisons between VACV-WR, DPP15, CPXV, and scHPXV YFP-gpt::095. BSC-40 cells were infected with the indicated viruses and at 48 h post infection the cells were fixed and stained. The areas (in arbitrary units [A.U.]) of 24 plaques over three independent experiments were measured for each virus. Data are expressed as the mean plaque diameter. **, P<0.01; ****, P<0.0001. C. FIG. 6C depicts plaque morphology of BSC-40 cells infected with the indicated viruses for 72 h. Cells were fixed, stained, and scanned for visualization.

FIG. 7. A graphical representation of the % weight loss overtime after administration of various compositions and doses to mice. The depicted data are generated from groups of 5 female BALB/c mice that are inoculated with the indicated dose of scHPXV YFP-gpt::095 (also designated as scHPXV(ΔHPXV_095/J2R) or scHPXV (yfp/gpt)), scHPXV (wt), Dryvax DPP15, or VACV WR in 10 μl of PBS. Mice are weighed daily for 28 days and any that lost >25% of their initial weight are euthanized. Data points represent mean scores, and error bars represent standard deviation.

FIGS. 8A and 8B. Graphical representations of the % weight loss over time after administration of various compositions and doses to mice. The depicted data are generated from mice that are previously vaccinated (FIG. 10) and who are then challenged with a lethal dose of VACV WR (10⁶ PFU) intranasally. FIG. 8A shows the weight changes and FIG. 8B shows the clinical scores in mice recorded daily for 13 days. Any mice that lost >25% of their initial weight are euthanized. Mice are assigned a clinical score based upon the appearance of ruffled fur, hunched posture, difficulty breathing, and decreased mobility. Data points represent mean differences in weights or scores, and error bars represent standard deviation. † indicate the number of mice that succumb to the VACV infection on a given day.

FIG. 9. Graphical representation of the % survival over time after administration of various compositions and doses to mice. The depicted data are generated from mice that are previously vaccinated (FIG. 7) and who are then challenged with a lethal dose of VACV WR (10⁶ PFU) intranasally. FIG. 9 shows survival curves of mice who are challenged intranasally with a lethal dose of VACV WR (10⁶ PFU). † indicate the number of mice that succumb to the VACV infection on the indicated day.

FIGS. 10A and 10B. Characterization of VACV-HPXV hybrid viruses. FIG. 10A. HPXV inserts in VACV strain WR. Virus genomes were sequenced using an Illumina platform, assembled, and LAGAN and “Base-by-Base” software were used to align and generate the maps shown. Places where VACV sequences (white) have been replaced by HPXV sequences are color coded according to the difference. FIG. 10B. A PCR-based screening approach for identifying hybrid and reactivated viruses. Following PCR amplification, the products were digested with BsaI to differentiate VACV sequences (which cut) from HPXV (which do not cut). The VACV/HPXV hybrids exhibit a mix of BsaI sensitive and resistant sites whereas the reactivated scHPXV YFP-gpt::095 clone is fully BsaI resistant.

FIG. 11A-11C. Growth properties of scHPXV versus scHPXV YFP-gpt::095. FIG. 11A. Plaque size measurements. Homologous recombination was used to replace the YFP-gpt locus in scHPXV YFP-gpt::095 with thymidine kinase gene sequences. This produced a virus with a fully wild-type complement of HPXV genes (scHPXV). BSC-40 cells were infected with the indicated viruses and cultured for three days. The dishes were stained and the plaque areas measured using a scanned digital image. Statistically significant differences are noted ****P<0.0001). FIG. 11B. Plaque images. FIG. 11C. Multi-step virus growth in culture. The indicated cell lines were infected with scHPXV or scHPXV YFP-gpt::095 at a multiplicity of infection of 0.01, the virus harvested at the indicated times, and titrated on BSC-40 cells in triplicate. No significant differences in the growth of these viruses were detected in these in vitro assays.

FIG. 12. Schematic representation of the linear dsDNA VACV genome strain ACAM2000; Genbank Accession AY313847. FIG. 12 depicts the modified VACV ACAM2000 genome that was used to chemically synthesize large ds DNA fragments. The overlapping scVACV ACAM2000 genomic fragments are depicted in blue. The engineered BsaI restriction sites that were not silently mutated in the Left Inverted Terminal Repeat (LITR) and the Right Inverted Terminal Repeat (RITR), are also shown.

FIG. 13A-13C. Detailed schematic representation of the first ˜1500-3000 bp of the published genomes of (A) VACV WR strain and (B) VACV ACAM2000. The tandem repeat regions are clearly indicated in red (70 bp repeat), blue (125 bp repeat) and green (54 bp repeat) boxes. The ORF corresponding to gene C23L is also indicated in each of the genomes. (C) Schematic representation of the direct repeat region containing 70 bp repeat sequences in VACV WR. This sequence was synthesized to contain a SapI restriction site at the 5′ terminus and an NheI restriction site at the 3′ terminus to ligate the hairpin/duplex piece and the VACV ACAM2000 ITR fragments, respectively.

FIG. 14. Assembly of vaccinia virus terminal hairpin loop with duplex DNA to the first 70 bp repeat sequence. Gel electrophoresis of duplex DNA (lane 2) and hairpin DNA alone (lane 3) and following ligation (lane 4) are depicted. The ligated product (arrow) was subsequently excised from the gel and purified, so that it could be ligated to a 70 bp repeat sequence to mimic the sequence of the wtVACV ACAM2000 sequence.

FIG. 15. Growth properties of scVACV ACAM2000-WR DUP/HP in vitro. Multi-step growth kinetics measured in monkey kidney epithelial cells (BSC-40). The cells were infected at a multiplicity of infection 0.03, the virus was harvested at the indicated times, and the virus was titrated on BSC-40 cells. The data represent three independent experiments. The error bars indicate standard error of the mean (SEM).

FIG. 16. Growth properties of scVACV ACAM2000-WR DUP/HP and scVACV ACAM2000-ACAM2000 DUP/HP in vitro, compared to scVACV ACAM2000-WR DUP/HP and scVACV ACAM2000-ACAM2000 DUP/HP where the YFP-gpt marker has been replaced with the J2R gene sequence (VAC_WRAJ2R) and wtVACV ACAM2000. Multi-step growth kinetics measured in monkey kidney epithelial cells (BSC-40). The cells were infected at a multiplicity of infection 0.03, the virus was harvested at the indicated times, and the virus was titrated on BSC-40 cells. The error bars indicate standard error of the mean (SEM).

FIG. 17. Restriction endonuclease mapping of reactivated scVACV ACAM2000-WR DUP/HP clones. Pulsed field gel electrophoretic analysis. Two independent scVACV ACAM2000-WR DUP/HP clones plus a VACV WR control, where the YFP-gpt marker has been replaced with the J2R gene sequence (VAC_WRAJ2R) and a wtVACV ACAM2000 control (VAC_ACAM2000) were purified and then left either undigested, digested with BsaI, HindIII, or Not and PvuI. The expected absence of nearly all of the BsaI sites in the scVACV ACAM2000 clones was apparent. Minor differences in the HindIII digested scVACV ACAM2000 genomic DNA compared to VACV_WRAJ2R and VACV_ACAM2000 were observed. Genomic DNA digested with Not and PvuI excises the 70 bp tandem repeat fragments found at the left and right ITR sequences. In VACV_WRAJ2R the ˜size of the 70 bp repeats is close to 3.6 kbp. Interestingly, in the two independent scVACV ACAM2000 clones two different sized bands corresponding to the 70 bp tandem repeat were observed (marked with an *), even though a full-length 70 bp tandem repeat element was ligated to the ITR fragments. When ACAM2000 genomic DNA was digested with Nod and PvuI, a band at ˜4.7 kbp was observed, which may indicate the size of the 70 bp repeats in ACAM2000.

### DETAILED DESCRIPTION OF THE DISCLOSURE

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of various aspects of the present invention will employ, unless otherwise indicated, conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Exemplary methods and materials are described herein, although methods and materials similar or equivalent to those described herein can also be used in the practice or testing of the present invention. The materials, methods, and examples are illustrative only and not intended to be limiting.

### Definitions

The following terms, unless otherwise indicated, shall be understood to have the following meanings:

As used herein, the terms “wild type virus”, “wild type genome”, “wild type protein,” or “wild type nucleic acid” refer to a sequence of amino or nucleic acids that occurs naturally within a certain population (e.g., a particular viral species, etc.).

The terms “chimeric” or “engineered” or “modified” (e.g., chimeric poxvirus, engineered polypeptide, modified polypeptide, engineered nucleic acid, modified nucleic acid) or grammatical variations thereof are used interchangeably herein to refer to a non-native sequence that has been manipulated to have one or more changes relative a native sequence.

As used herein, “synthetic virus” refers to a virus initially derived from synthetic DNA (e.g., chemically synthesized DNA, PCR amplified DNA, engineered DNA, polynucleotides comprising nucleoside analogs, etc., or combinations thereof) and includes its progeny, and the progeny may not necessarily be completely identical (in morphology or in genomic DNA complement) to the original parent synthetic virus due to natural, accidental, or deliberate mutation. In some embodiments, the synthetic virus refers to a virus where substantially all of the viral genome is initially derived from synthetic DNA (e.g., chemically synthesized DNA, PCR amplified DNA, engineered DNA, polynucleotides comprising nucleoside analogs, etc., or combinations thereof). In a preferred embodiment, the synthetic virus is derived from chemically synthesized DNA.

As outlined elsewhere herein, certain positions of the viral genome can be altered. By “position” as used herein is meant a location in the genome sequence. Corresponding positions are generally determined through alignment with other parent sequences.

As used herein, the term “residue” in the context of a polypeptide refers to an amino-acid unit in the linear polypeptide chain. It is what remains of each amino acid, i.e —NH—CHR—C—, after water is removed in the formation of the polypeptide from α-amino-acids, i.e. NH2-CHR—COOH.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog; internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.); those containing pendant moieties, such as, for example, proteins (e.g., nucleases, toxins, antibodies, signal peptides, poly-L-lysine, etc.); those with intercalators (e.g., acridine, psoralen, etc.); those containing chelators (e.g., metals, radioactive metals, boron, oxidative metals, etc.); those containing alkylators; those with modified linkages (e.g., alpha anomeric nucleic acids, etc.); as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. “Homologous” may also refer to a nucleic acid which is native to the virus.

However, in common usage and in the instant application, the term “homologous,” when modified with an adverb such as “highly,” may refer to sequence similarity and may or may not relate to a common evolutionary origin.

“Heterologous,” in all its grammatical forms and spelling variations, may refer to a DNA which is non-native to the virus. It means derived from a different species or a different strain than the DNA of the organism to which the DNA is described as heterologous relative to. In a non-limiting example, the viral genome of the scPV comprises heterologous terminal hairpin loops. Said heterologous terminal hairpin loops can be derived from a different virus species or from a different virus strain.

The term “sequence similarity,” in all its grammatical forms, refers to the degree of identity or correspondence between nucleic acid or amino acid sequences that may or may not share a common evolutionary origin.

“Percent (%) sequence identity” or “sequence % identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected and/or transformed in vivo with a nucleic acid of this disclosure.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature. Thus, a molecule that is chemically synthesized, or expressed in a cellular system different from the cell from which it naturally originates, will be “isolated” from one or many of its naturally associated components. A molecule also may be rendered substantially free of from one or many of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, the term “isolated”, in the context of viruses, refers to a virus that is derived from a single parental virus. A virus can be isolated using routine methods known to one of skill in the art including, but not limited to, those based on plaque purification and limiting dilution.

As used herein, the phrase “multiplicity of infection” or “MO” is the average number of viruses per infected cell. The MOI is determined by dividing the number of virus added (ml added×plaque forming units (PFU)) by the number of cells added (ml added×cells/ml).

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition). As used herein “purified” in the context of viruses refers to a virus which is substantially free of cellular material and culture media from the cell or tissue source from which the virus is derived. The language “substantially free of cellular material” includes preparations of virus in which the virus is separated from cellular components of the cells from which it is isolated or recombinantly produced. Thus, virus that is substantially free of cellular material includes preparations of protein having less than about 30%, 20%, 10%, or 5% (by dry weight) of cellular protein (also referred to herein as a “contaminating protein”). The virus is also substantially free of culture medium, i.e., culture medium represents less than about 20%, 10%, or 5% of the volume of the virus preparation. A virus can be purified using routine methods known to one of skill in the art including, but not limited to, chromatography and centrifugation.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient”, “subject”, or “individual” are used interchangeably herein and refer to either a human or a non-human animal. These terms include mammals, such as humans, primates, livestock animals (including bovines, porcines, camels, etc.), companion animals (e.g., canines, felines, etc.) and rodents (e.g., mice and rats).

As used herein, the terms “prevent”, “preventing” and “prevention” refer to the delay of the recurrence or onset of disease, or a reduction in one or more symptoms of a disease (e.g., a poxviral infection) in a subject as a result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of an infection (e.g., a poxviral infection or a condition associated therewith), or the prevention of the recurrence, onset, or development of one or more symptoms of an infection (e.g., a poxviral infection or a condition associated therewith), in a subject resulting from the administration of a therapy (e.g., a prophylactic or therapeutic agent), or the administration of a combination of therapies (e.g., a combination of prophylactic or therapeutic agents).

As used herein, the terms “treat”, “treating” or “treatment” refer to treating a condition or patient and refers to taking steps to obtain beneficial or desired results, including clinical results. With respect to infections (e.g., a poxviral infection or a variola virus infection), treatment refers to the eradication or control of the replication of an infectious agent (e.g., the poxvirus or the variola virus), the reduction in the numbers of an infectious agent (e.g., the reduction in the titer of the virus), the reduction or amelioration of the progression, severity, and/or duration of an infection (e.g., a poxviral/variola infection or a condition or symptoms associated therewith), or the amelioration of one or more symptoms resulting from the administration of one or more therapies (including, but not limited to, the administration of one or more prophylactic or therapeutic agents). With respect to cancer, treatment refers to the eradication, removal, modification, or control of primary, regional, or metastatic cancer tissue that results from the administration of one or more therapeutic agents of the disclosure. In certain embodiments, such terms refer to minimizing or delaying the spread of cancer resulting from the administration of one or more therapeutic agents of the invention to a subject with such a disease. In other embodiments, such terms refer to elimination of disease-causing cells.

“Administering” or “administration of” a substance, a compound or an agent to a subject can be carried out using one of a variety of methods known to those skilled in the art. For example, a compound or an agent can be administered sublingually or intranasally, by inhalation into the lung or rectally. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Each embodiment described herein may be used individually or in combination with any other embodiment described herein.

### Overview

Poxviruses are large (˜200 kbp) DNA viruses that replicate in the cytoplasm of infected cells. The Orthopoxvirus (OPV) genus comprises a number of poxviruses that vary greatly in their ability to infect different hosts. Vaccinia virus (VACV), for example, can infect a broad group of hosts, whereas variola virus (VARV), the causative agent of smallpox, only infects humans. A feature common to many, if not all poxviruses, is their ability to non-genetically “reactivate” within a host. Non-genetic reactivation refers to a process wherein cells infected by one poxvirus can promote the recovery of a second “dead” virus (for example one inactivated by heat) that would be non-infectious on its own.

Purified poxvirus DNA is not infectious because the virus life cycle requires transcription of early genes via the virus-encoded RNA polymerases that are packaged in virions. However, this deficiency can be overcome if virus DNA is transfected into cells previously infected with a helper poxvirus, providing the necessary factors needed to transcribe, replicate, and package the transfected genome in trans (Sam C K, Dumbell K R. Expression of poxvirus DNA in coinfected cells and marker rescue of thermosensitive mutants by subgenomic fragments of DNA. Ann Virol (Inst Past). 1981; 132:135-50). Although this produces mixed viral progeny, the problem can be overcome by performing the reactivation reaction in a cell line that supports the propagation of both viruses, and then eliminating the helper virus by plating the mixture of viruses on cells that do not support the helper virus' growth (Scheiflinger F, Dorner F, Falkner F G. Construction of chimeric vaccinia viruses by molecular cloning and packaging. Proceedings of the National Academy of Sciences of the United States of America. 1992; 89(21):9977-81).

Previously, Yao and Evans described a method in which the high-frequency recombination and replication reactions catalyzed by a Leporipoxvirus, Shope fibroma virus (SFV), can be coupled with an SFV-catalyzed reactivation reaction, to rapidly assemble recombinant vaccinia strains using multiple overlapping fragments of viral DNA (Yao X D, Evans D H. High-frequency genetic recombination and reactivation of orthopoxviruses from DNA fragments transfected into leporipoxvirus-infected cells. Journal of Virology. 2003; 77(13):7281-90).

### Stem Cells of the Disclosure

One aspect of the invention provides stem cells comprising a synthetic chimeric poxvirus (scPV), which can be used for the treatment of cancer and infectious diseases. The functional synthetic chimeric poxvirus (scPV) comprised within the stem cells, is initially replicated and assembled from chemically synthesized DNA. The scPV can be any poxvirus whose genome has been sequenced or can be sequenced in large part or for which a natural isolate is available. The viruses that may be produced in accordance with various embodiments of the methods of the invention can be any poxvirus whose genome has been sequenced in large part or for which a natural isolate is available. In some aspects, an scPV of the invention may be based on the genome sequences of naturally occurring strains, variants or mutants, mutagenized viruses or genetically engineered viruses. In some aspects, the viral genome of an scPV of the invention comprises one or more modifications relative to the wild type genome or base genome sequence of said virus. The modifications may include, for example, one or more deletions, insertions, substitutions, or combinations thereof. It is understood that the modifications may be introduced in any number of ways commonly known in the art.

As used herein, a “stem cell” is any totipotent, pluripotent or multipotent cell that has the ability to differentiate into multiple different types of cells (e.g., terminally differentiated cells). The term “stem cell”, as used herein, refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this invention. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

Any stem cell type can be used in the various aspects of the present invention. Stem cells include any type of stem cell, including embryonic stem cells (ES) cells, post-natal stem cells (e.g. from the umbilical cord and placenta), fetal stem cells and adult stem cells (i.e., somatic stem cells). Other types, such as induced pluripotent stem cells (iPSCs), are produced in the lab by reprogramming adult cells to express ES characteristics. In one embodiment, the adult stem cells are mesenchymal stem cells. In one embodiment, the adult stem cells are tissue or organ specific stem cells such as neuronal stem cells, vascular stem cells, or epidermal stem cells. In a preferred embodiment, the adult stem cells are mesenchymal stem cells (MSC).

Mesenchymal stem cells can be obtained from a variety of sources such as bone marrow, umbilical cord blood, and adipose tissue (adipose-tissue derived stem cells). Common sources of stem cells are human umbilical vein endothelial cells (HUVEC), and primary human cutaneous microvascular endothelial cells (HCMEC). Analogous non-human stem cells can be obtained from similar non-human sources as well. Stem cells for use in one aspect of the invention may be primary cells or cells that have been maintained in cell culture for an extended period. The stem cells may be obtained from any animal type, including human. In a preferred embodiment, the stem cell is a human cell.

As used herein, “embryonic stem cells” are stem cells obtained from an embryo that is typically six weeks old or less. Totipotent human embryonic stem cells (hESC) generally can be obtained from embryos that are 5 to 7 days old. Pluripotent human primordial germ cells (hEG) typically can be obtained from embryos that are six weeks old or less. As use herein, “fetal stem cells” refer to any stem cell that is obtained prenatally from a fetus that is typically greater that 6 weeks old. As used herein, “adult stem cells” refers to any stem cell that is obtained from a post-natal subject. Typically, the subject is a full grown adult. Exemplary adult stem cells include, but are not limited to, cells harvested from organs such as fat, muscle or bone marrow.

In one embodiment, the stem cells are autologous, i.e., the cells are obtained or derived from the subject's own stem cells. In one embodiment, the stem cells of the invention are obtained or derived from a subject who is in need of therapeutic treatment for a cell proliferative disorder (tumor or cancer). The subject may already have the cell proliferative disorder or be at risk for the disorder.

In another embodiment, the stem cells are allogeneic, i.e., the cells are obtained or derived from a donor whose human leukocyte antigens (HLA) are acceptable matches to the subject's.

In one aspect, the invention relates to an isolated stem cell or population thereof comprising a synthetic chimeric poxvirus (scPV), wherein the virus is replicated and reactivated from DNA derived from synthetic DNA, the viral genome of said virus differing from a wild type genome of said virus in that it is characterized by one or more modifications.

In another aspect, the invention relates to an isolated stem cell or population thereof comprising a synthetic chimeric orthopoxvirus, wherein the virus is replicated and reactivated from DNA derived from synthetic DNA, the viral genome of said virus differing from a wild type genome of said virus in that it is characterized by one or more modifications.

In another aspect, the invention relates to an isolated stem cell or population thereof comprising a synthetic chimeric vaccinia virus, wherein the virus is replicated and reactivated from DNA derived from synthetic DNA, the viral genome of said virus differing from a wild type genome of said virus in that it is characterized by one or more modifications.

In one aspect, the invention relates to an isolated stem cell or population thereof comprising a synthetic chimeric horsepox virus, wherein the virus is replicated and reactivated from DNA derived from synthetic DNA, the viral genome of said virus differing from a wild type genome of said virus in that it is characterized by one or more modifications.

In one embodiment, the stem cell of the invention is a non-cancer stem cell.

### Synthetic Chimeric Poxviruses of the Disclosure

Chemical genome synthesis is particularly useful when a natural template is not available for genetic modification, amplification, or replication by conventional molecular biology methods. For example, a natural isolate of horsepox virus (HPXV) is not readily available to obtain template DNA but the genome sequence for HPXV (strain MNR-76) has been described. The HPXV genome sequence, however, is incomplete. The sequence of the terminal hairpin loops was not determined. Therefore, a functional synthetic chimeric HPXV (scHPXV) can be generated by using terminal hairpin loops based on VACV telomeres in lieu of HPXV terminal hairpin loop sequences. Similarly, the genome sequence for wtVACV (strain NYCBH, clone ACAM2000) has been described and published, though it is not complete. The sequence of the terminal hairpin loops was not determined, only four 54 bp repeat sequences were identified. Therefore, a functional synthetic chimeric VACV ACAM2000 can be generated by using terminal hairpin loops based on a different strain of VACV (such as WR strain) in lieu of VACV ACAM2000 terminal hairpin loop sequences. In other embodiments, the terminal hairpin loops are based on the VACV ACAM2000 terminal hairpin loop sequences.

In some embodiments, the poxvirus belongs to the Chordopoxvirinae subfamily. In some embodiments, the poxvirus belongs to a genus of Chordopoxvirinae subfamily selected from Avipoxvirus, Capripoxvirus, Cervidpoxvirus, Crocodylipoxvirus, Leporipoxvirus, Molluscipoxvirus, Orthopoxvirus, Parapoxvirus, Suipoxvirus, or Yatapoxvirus. In some embodiments, the poxvirus is an Orthopoxvirus. In some embodiments, the Orthopoxvirus is selected from camelpox virus (CMLV), cowpox virus (CPXV), ectromelia virus (ECTV, “mousepox agent”), HPXV, monkeypox virus (MPXV), rabbitpox virus (RPXV), raccoonpox virus, skunkpox virus, Taterapox virus, Uasin Gishu disease virus, vaccinia virus (VACV), variola virus (VARV) and volepox virus (VPV). In a preferred embodiment, the poxvirus is an HPXV. In another preferred embodiment, the poxvirus is a VACV. In some embodiments, the poxvirus is a Parapoxvirus. In some embodiments, the Parapoxvirus is selected from orf virus (ORFV), pseudocowpox virus (PCPV), bovine popular stomatitis virus (BPSV), squirrel parapoxvirus (SPPV), red deer parapoxvirus, Ausdyk virus, Chamois contagious ecythema virus, reindeer parapoxvirus, or sealpox virus. In some embodiments, the poxvirus is a Molluscipoxvirus. In some embodiments, the Molluscipoxvirus is molluscum contagiousum virus (MCV). In some embodiments, the poxvirus is a Yatapoxvirus. In some embodiments, the Yatapoxvirus is selected from Tanapox virus or Yaba monkey tumor virus (YMTV). In some embodiments, the poxvirus is a Capripoxvirus. In some embodiments, the Capripoxvirus is selected from sheepox, goatpox, or lumpy skin disease virus. In some embodiments, the poxvirus is a Suipoxvirus. In some embodiments, the Suipoxvirus is swinepox virus. In some embodiments, the poxvirus is a Leporipoxvirus. In some embodiments, the Leporipoxvirus is selected from myxoma virus, Shope fibroma virus (SFV), squirrel fibroma virus, or hare fibroma virus. New poxviruses (e.g., Orthopoxviruses) are still being constantly discovered. It is understood that an scPV of the various aspects of the invention may be based on such a newly discovered poxvirus.

In some aspects, the scPV is a CMLV whose genome is based on a published genome sequence (e.g., strain CMS (Genbank Accession AY009089.1)). In some aspects, the scPV is a CPXV whose genome is based on a published genome sequence (e.g., strain Brighton Red (Genbank Accession AF482758), strain GRI-90 (Genbank Accession X94355)). In some aspects, the scPV is a ECTV whose genome is based on a published genome sequence (e.g. strain Moscow (Genbank Accession NC_004105)). In some aspects, the scPV is a MPXV whose genome is based on a published genome sequence (e.g., strain Zaire-96-1-16 (Genbank Accession AF380138)). In some aspects, the scPV is a RPXV whose genome is based on a published genome sequence (e.g. strain Utrecht (Genbank Accession AY484669)). In some aspects, the scPV is a Taterapox virus whose genome is based on a published genome sequence (e.g., strain Dahomey 1968 (Genbank Accession NC_008291)).

Chemical viral genome synthesis also opens up the possibility of introducing a large number of useful modifications to the resulting genome or to specific parts of it. The modifications may improve ease of cloning to generate the virus, provide sites for introduction of recombinant gene products, improve ease of identifying reactivated viral clones and/or confer a plethora of other useful features (e.g., introducing a desired antigen, producing an oncolytic virus, etc.). In some embodiments, the modifications may include the attenuation or deletion of one or more virulence factors. In some embodiments, the modifications may include the addition or insertion of one or more virulence regulatory genes or gene-encoding regulatory factors.

In one aspect, the invention provides polynucleotides for producing a synthetic chimeric horsepox virus (scHPXV). In a specific embodiment, the scHPXV genome may be based on the genome sequence described for HPXV strain MNR-76 (SEQ ID NO: 49) (Tulman E R, Delhon G, Afonso C L, Lu Z, Zsak L, Sandybaev N T, et al. Genome of horsepox virus. Journal of Virology. 2006; 80(18):9244-58). This genome sequence is incomplete and appears not to include the sequence of the terminal hairpin loops. It is shown here that terminal hairpin loops from vaccinia virus (VACV) can be ligated onto the ends of the HPXV genome to produce functional scHPXV particles using the methods of the invention. The HPXV genome may be divided into 10 overlapping fragments as described in the working examples of the disclosure and shown in Table 1. In some embodiments, the genome may be divided into 2, 3, 4, 5, 6, 7, 8, 9, 10, 11, 12, 13, 14, or 15 overlapping fragments. In some embodiments, the entire genome may be provided as one fragment. The genomic locations of the exemplary overlapping fragments and fragment sizes are shown in Table 1. Table 2 shows some of the modifications that may be made in these fragments relative to the base sequence. The polynucleotides of one aspect of the invention comprise nucleic acids sequences that are at least 65%, 70%, 75%, 80%, 85%, 90%, 91%, 92%, 93%, 94%, 95%, 96%, 97%, 98%, 99%, or 100% identical to SEQ ID NOs: 1-10. In some embodiments, an isolated polynucleotide of the invention comprises a variant of these sequences, wherein such variants can include missense mutations, nonsense mutations, duplications, deletions, and/or additions. SEQ ID NO: 11 and SEQ ID NO: 12 depict the nucleotide sequences of VACV (WR strain) terminal hairpin loops. In some embodiments, the terminal hairpin loops comprise nucleic acid sequences that are at least 70%, 75%, 80%, 85%, 90%, 91%, 92%, 93%, 94%, 95%, 96%, 97%, 98%, 99%, or 100% identical to SEQ ID NO: 11 or SEQ ID NO: 12.

In another aspect, the invention provides polynucleotides for producing a synthetic chimeric vaccinia virus (scVACV). In a specific embodiment, the scVACV genome may be based on the published genome sequence described for VACV strain NYCBH clone ACAM2000 (GenBank accession AY313847; Osborne J D et al. Vaccine. 2007; 25(52):8807-32). This genome sequence is incomplete and appears not to include the sequence of the terminal hairpin loops and only four 54 bp repeat sequences were identified. It is shown in the present application that terminal hairpin loops from vaccinia virus (VACV) strain WR can be ligated onto the ends of the VACV genome strain NYCBH clone ACAM2000 to produce functional scVACV particles using the methods of the disclosure. In some embodiments, the terminal hairpin loops from vaccinia virus (VACV) strain ACAM2000 can be ligated onto the ends of the VACV genome strain NYCBH clone ACAM2000 to produce functional scVACV particles using the methods of the disclosure. The scVACV genome may be divided into 9 overlapping fragments as described in the working examples of the disclosure and shown in Table 4. In some embodiments, the VACV genome may be divided into 2, 3, 4, 5, 6, 7, 8, 9, 10, 11, 12, 13, 14, or 15 overlapping fragments. In some embodiments, the entire genome may be provided as one fragment. The fragment sizes are shown in Table 4. The polynucleotides of one aspect of the invention comprise nucleic acids sequences that are at least 65%, 70%, 75%, 80%, 85%, 90%, 91%, 92%, 93%, 94%, 95%, 96%, 97%, 98%, 99%, or 100% identical to SEQ ID NOs: 54-62. In some embodiments, an isolated polynucleotide of the invention comprises a variant of these sequences, wherein such variants can include missense mutations, nonsense mutations, duplications, deletions, and/or additions. SEQ ID NO: 11 and SEQ ID NO: 12 depict the nucleotide sequences of VACV (WR strain) terminal hairpin loops. SEQ ID NO: 117 and SEQ ID NO: 118 depict the nucleotide sequences of VACV (ACAM2000 strain) terminal hairpin loops. In some embodiments, the terminal hairpin loops comprise nucleic acid sequences that are at least 70%, 75%, 80%, 85%, 90%, 91%, 92%, 93%, 94%, 95%, 96%, 97%, 98%, 99%, or 100% identical to SEQ ID NO: 11 or to SEQ ID NO: 12. In some embodiments, the terminal hairpin loops comprise nucleic acid sequences that are at least 70%, 75%, 80%, 85%, 90%, 91%, 92%, 93%, 94%, 95%, 96%, 97%, 98%, 99%, or 100% identical to SEQ ID NO: 117 or to SEQ ID NO: 118.

Traditionally, the terminal hairpins of poxviruses have been difficult to clone and sequence, hence, it is not surprising that some of the published genome sequences (e.g., VACV, ACAM 2000 and HPXV MNR-76) are incomplete. The published sequence of the HPXV genome is likewise incomplete, probably missing ˜60 bp from the terminal ends. Thus, the HPXV hairpins cannot be precisely replicated. In an exemplary embodiment, 129 nt ssDNA fragments were chemically synthesized using the published sequence of the VACV terminal hairpins as a guide and ligated onto dsDNA fragments comprising left and right ends of the HPXV genome. Likewise, the genome sequence for wtVACV, strain NYCBH, clone ACAM2000, has been described and published, though it is not complete. The sequence of the terminal hairpin loops was not determined, only four 54 bp repeat sequences were identified. Since the published sequence of the wtVACV strain NYCBH, clone ACAM2000 genome is incomplete, the hairpins cannot be precisely replicated. In an exemplary embodiment, ssDNA fragments were chemically synthesized using the published sequence of the wtVACV WR strain terminal hairpin loops as a guide and ligated onto dsDNA fragments comprising left and right ends of the VACV strain NYCBH.

In another embodiment, the viral genome of the scPV of the present disclosure comprises homologous or heterologous terminal hairpin loops and the tandem repeat regions (the 70 bp, the 125 bp and the 54 bp tandem repeats) located downstream of the hairpin loops, wherein the tandem repeat regions comprise a different number of repeats than the wtVACV (i.e. the virus present in nature). The number of repeats of the 70 bp, the 125 bp and the 54 bp tandem repeats found in the VACV virus, strain WR were 22, 2 and 8, respectively. In another embodiment, the number of tandem repeat regions are variable in the different poxviruses, in the different vaccinia viruses or in the different vaccinia virus strains. The term “homologous terminal hairpin loops” means that said terminal hairpin loops are coming from the same virus species/the same strain, while the term “heterologous terminal hairpin loops” means that said terminal hairpin loops are coming from a different virus species/different strain.

In some embodiments, the terminal hairpins of an scPV of the invention are derived from VACV. In some embodiments, the terminal hairpins are derived from CMLV, CPXV, ECTV, HPXV, MPXV, RPXV, raccoonpox virus, skunkpox virus, Taterapox virus, Uasin Gishu disease virus or VPV. In some embodiments, the terminal hairpin loops are based on a strain selected from the group of: Western Reserve, Clone 3, Tian Tian, Tian Tian clone TP5, Tian Tian clone TP3, NYCBH, NYCBH clone Acambis 2000, Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), IHD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Chorioallantois Vaccinia virus Ankara (CVA), Modified vaccinia Ankara (MVA), and MVA-BN. In one embodiment, the terminal hairpin loops are based on the Western Reserve strain (WR strain) of VACV. In another embodiment, the terminal hairpin loops are based on the ACAM2000 strain. New VACV strains are still being constantly discovered. It is understood that an scPV of the various aspects of the invention may be based on such a newly discovered poxviruses or newly discovered strains.

In some embodiments, the modifications may include the deletion of one or more restriction sites. In some embodiments, the modifications may include the introduction of one or more restriction sites. In some embodiments, the restriction sites to be deleted from the genome or added to the genome may be selected from one or more of restriction sites such as but not limited to AanI, AarI, AasI, AatI, AatII, AbaSI, AbsI, Acc65I, AccI, AccI, AccIII, Aci, AcII, AcuI, AfeI, AflII, AfIHI, AgeI, AhdI, AeI, AluI, AwI, ALwNI, ApaI, ApaLI, ApeKI, ApoI, AscI, AseI, AsiSI, AvaI, AvaII, AvrII, BaeGI, BaeI, BamHI BanI, BanI, BbsI, BbvCI, BbvI, BccI, BceAI, BcgI, BciVI, BcI, BcoDI, BfaI, BfuAI, BfuCI, BglI, BglII, BlpI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BsI, BsmAI, BsmBI, BsmFI, BsmI, BsoBI, Bsp1286I, BspCNI, BspDI, BspEI, BspHI, BspMI, BspQI, BsrBI, BsrDI, BsrFa, BsrGI, BsrI, BssHII, BssSa, BstAPI, BstBI, BstEII, BstNI, BstUI, BstX, BstYI, BstZ17I, Bsu36I, BtgI, BtgZI, Btsa, BtsCI, BtsIMutI, Cac8I, ClaI, CspCI, CviAII, CviKI-1, CviQI, DdeI, DpnI, DpnII, DraI, DrdI, EaeI, EagI, EarI, EciI, Eco53kI, EcoNI, EcoO109I, EcoPI5I, EcoRI, EcoRV, FatI, FauI, Fnu4HI, FokI, FseI, FspEI, FspI, HaeII, HaeIII, HgaI, HhaI, HincI, Hindll, Hinfl, HinPl, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, LpnPI, MboI, MboII, MfeI, MuCI, MZuI, MyI, MmeI, MnlI, MscI, MseI, MsI, MspA, MspI, MspJI, MwoI, NaeI, NarI, NciI, NcoI, NdeI, NgoMIV, NheI, NaIII, NaIV, NmeAIII, NotI, NruI, NsiI, NspI, PacI, PaeR7I, PciI, PflFI, PflMI, PleI, PluTI, PmeI, PmI, PpuMI, PshAI, PsiI, PspGI, PspOMI, PspXI, PstI, PvuI, PvuII, RsaI, RsrII, SacI, SacI, SaI, SapI, Sau3AI, Sau96I, Sbfl, ScrFI, SexAI, SfaNI, SfcI, SfiI, SfoI, SgrAI, SmaI, SmlI, SnaBI, SpeI, SphI, SrfI, SspI, StuI, StyD4I, StyI, SwaI, Taqa, TfI, TseI, Tsp45I, TspMI, TspRI, Tth111I, XbaI, XcmI, XhoI, XmaI, XmnI, or ZraI. It is understood that any desired restriction site(s) or combination of restriction sites may be inserted into the genome or mutated and/or eliminated from the genome. In some embodiments, one or more AarI sites are deleted from the viral genome. In some embodiments, one or more BsaI sites are deleted from the viral genome. In some embodiments, one or more restriction sites are completely eliminated from the genome (e.g., all the AarI sites in the viral genome may be eliminated). In some embodiments, one or more AvaI restriction sites are introduced into the viral genome. In some embodiments, one or more Stu sites are introduced into the viral genome. In some embodiments, the one or more modifications may include the incorporation of recombineering targets including, but not limited to, loxP or FRT sites.

In some embodiments, the modifications may include the introduction of fluorescence markers such as, but not limited to, green fluorescent protein (GFP), enhanced GFP, yellow fluorescent protein (YFP), cyan/blue fluorescent protein (BFP), red fluorescent protein (RFP), or variants thereof, etc.; selectable markers such as but not limited to drug resistance markers (e.g., E. coli xanthine-guanine phosphoribosyl transferase gene (gpt), Streptomyces alboniger puromycin acetyltransferase gene (pac), neomycin phosphotransferase I gene (nptI), neomycin phosphotransferase gene II (nptII), hygromycin phosphotransferase (hpt), sh ble gene, etc.; protein or peptide tags such as but not limited to MBP (maltose-binding protein), CBD (cellulose-binding domain), GST (glutathione-S-transferase), poly(His), FLAG, V5, c-Myc, HA (hemagglutinin), NE-tag, CAT (chloramphenicol acetyl transferase), DHFR (dihydrofolate reductase), HSV (Herpes simplex virus), VSV-G (Vesicular stomatitis virus glycoprotein), luciferase, protein A, protein G, streptavidin, T7, thioredoxin, Yeast 2-hybrid tags such as B42, GAL4, LexA, or VP16; localization tags such as an NLS-tag, SNAP-tag, Myr-tag, etc. It is understood that other selectable markers and/or tags known in the art may be used. In some embodiments, the modifications include one or more selectable markers to aid in the selection of reactivated clones (e.g., a fluorescence marker such as YFP, a drug selection marker such as gpt, etc.) to aid in the selection of reactivated viral clones. In some embodiments, the one or more selectable markers are deleted from the reactivated clones after the selection step.

### Methods of Producing Synthetic Chimeric Poxviruses

The invention provides, in some aspects, systems and methods for synthesizing, reactivating and isolating functional synthetic chimeric poxviruses (scPVs) from chemically synthesized overlapping double-stranded DNA fragments of the viral genome. Recombination of overlapping DNA fragments of the viral genome and reactivation of the functional scPV are carried out in cells previously infected with a helper virus. Briefly, overlapping DNA fragments that encompass all or substantially all of the viral genome of the scPV are chemically synthesized and transfected into helper virus-infected cells. The transfected cells are cultured to produce mixed viral progeny comprising the helper virus and reactivated scPV. Next, the mixed viral progeny are plated on host cells that do not support the growth of the helper virus but allow the synthetic chimeric poxvirus to grow, in order to eliminate the helper virus and recover the synthetic chimeric poxvirus. In some embodiments, the helper virus does not infect the host cells. In some embodiments, the helper virus can infect the host cells but grows poorly in the host cells. In some embodiments, the helper virus grows more slowly in the host cells compared to the scPV.

In some embodiments, substantially all of the synthetic chimeric poxvirus genome is derived from chemically synthesized DNA. In some embodiments, about 40%, about 50%, about 60%, about 70%, about 75%, about 80%, about 85%, about 90%, about 95%, about 96%, about 97%, about 98%, about 99%, over 99%, or 100% of the synthetic chimeric vaccinia virus genome is derived from chemically synthesized DNA. In some embodiments, the poxvirus genome is derived from a combination of chemically synthesized DNA and naturally occurring DNA. In some embodiments, all of the fragments encompassing the vaccinia virus genome are chemically synthesized. In some embodiments, one or more of the fragments are chemically synthesized and one or more of the fragments are derived from naturally occurring DNA (e.g., by PCR amplification or by well-established recombinant DNA techniques).

The number of overlapping DNA fragments used in the various embodiments of the methods of the invention will depend on the size of the poxvirus genome, such as horsepox virus or vaccinia virus. Practical considerations such as reduction in recombination efficiency as the number of fragments increases on the one hand, and difficulties in synthesizing very large DNA fragments as the number of fragments decreases on the other hand, will also inform the number of overlapping fragments used in the methods of the invention. In some embodiments, the synthetic chimeric vaccinia virus genome may be synthesized as a single fragment. In some embodiments, the synthetic chimeric vaccinia virus genome is assembled from 2-14 overlapping DNA fragments. In some embodiments, the synthetic chimeric vaccinia virus genome is assembled from 4-12 overlapping DNA fragments. In some embodiments, the synthetic chimeric vaccinia virus genome is assembled from 6-12 overlapping DNA fragments. In some embodiments, the synthetic chimeric vaccinia virus genome is assembled from 8-11 overlapping DNA fragments. In some embodiments, the synthetic chimeric vaccinia virus genome is assembled from 8-10, 10-12, or 10-14 overlapping DNA fragments. In some embodiments, the synthetic chimeric vaccinia virus genome is assembled from 2, 3, 4, 5, 6, 7, 8, 9, 10, 11, 12, 13, 14, or 15 overlapping DNA fragments. In an exemplary embodiment, the synthetic chimeric vaccinia virus genome is assembled from 9 overlapping DNA fragments. In some embodiments, the synthetic chimeric horsepox virus genome is assembled from 2-14 overlapping DNA fragments. In some embodiments, the synthetic chimeric horsepox virus genome is assembled from 4-12 overlapping DNA fragments. In some embodiments, the synthetic chimeric horsepox virus genome is assembled from 6-12 overlapping DNA fragments. In some embodiments, the synthetic chimeric horsepox virus genome is assembled from 8-11 overlapping DNA fragments. In some embodiments, the synthetic chimeric horsepox virus genome is assembled from 8-10, 10-12, or 10-14 overlapping DNA fragments. In some embodiments, the synthetic chimeric horsepox virus genome is assembled from 2, 3, 4, 5, 6, 7, 8, 9, 10, 11, 12, 13, 14, or 15 overlapping DNA fragments. In an exemplary embodiment of the disclosure, a synthetic vaccinia virus (scVACV) is reactivated from 9 chemically synthesized overlapping double-stranded DNA fragments. In another exemplary embodiment of the disclosure, a synthetic chimeric horsepox virus (scHPXV) is reactivated from 10 chemically synthesized overlapping double-stranded DNA fragments. In some embodiments, terminal hairpin loops are synthesized separately and ligated onto the fragments comprising the left and right ends of the vaccinia virus genome. In some embodiments, terminal hairpin loops may be derived from a naturally occurring template. In some embodiments, the terminal hairpins of an scPV of the disclosure are derived from VACV. In some embodiments, the terminal hairpins are derived from CMLV, CPXV, ECTV, HPXV, MPXV, RPXV, raccoonpox virus, skunkpox virus, Taterapox virus, Uasin Gishu disease virus or VPV. In other embodiments, the terminal hairpins of an scVACV of the invention are derived from wtVACV. In some embodiments, the terminal hairpins are derived from wtVACV terminal hairpins of a different strain in lieu of the VACV own terminal hairpin loop sequences. In some embodiments, the terminal hairpins are based on the terminal hairpins of any wtVACV whose genome has been completely sequenced or a natural isolate of which is available for genome sequencing. In a preferred embodiment, the terminal hairpins are derived from VACV.

The size of the overlapping fragments used in the various embodiments of the methods of the invention will depend on the size of the poxvirus genome. It is understood that there can be wide variations in fragment sizes and various practical considerations, such as the ability to chemically synthesize very large DNA fragments, will inform the choice of fragment sizes. In some embodiments, the fragments range in size from about 2,000 bp to about 50,000 bp. In some embodiments, the fragments range in size from about 3,000 bp to about 45,000 bp. In some embodiments, the fragments range in size from about 4,000 bp to 40,000 bp. In some embodiments, the fragments range in size from about 5,000 bp to 35,000 bp. In some embodiments, the largest fragments are about 18,000 bp, 20,000 bp, 21,000 bp, 22,000 bp, 23,000 bp, 24,000 bp, 25,000 bp, 26,000 bp, 27,000 bp, 28,000 bp, 29,000 bp, 30,000 bp, 31,000 bp, 32,000 bp, 33,000 bp, 34,000 bp, 35,000 bp, 36,000 bp, 37,000 bp, 38,000 bp, 39,000 bp, 40,000 bp, 41,000 bp, 42,000 bp, 43,000 bp, 44,000 bp, 45,000 bp, 46,000 bp, 47,000 bp, 48,000 bp, 49,000 bp, or 50,000 bp. In an exemplary embodiment of the disclosure, an scVACV is reactivated from 9 chemically synthesized overlapping double-stranded DNA fragments ranging in size from about 10,000 bp to about 32,000 bp (Table 4). In an exemplary embodiment of the disclosure, an scHPXV is reactivated from 9 chemically synthesized overlapping double-stranded DNA fragments ranging in size from about 10,000 bp to about 32,000 bp (Table 1).

The helper virus may be any poxvirus that can provide the trans-acting enzymatic machinery needed to reactivate a poxvirus from transfected DNA. The helper virus may have a different or narrower host cell range than an scPV to be produced (e.g., Shope fibroma virus (SFV) has a very narrow host range compared to Orthopoxviruses such as vaccinia virus (VACV) or HPXV). The helper virus may have a different plaque phenotype compared to the scPV to be produced. In some embodiments, the helper virus is a Leporipoxvirus. In some embodiments, the Leporipoxvirus is an SFV, hare fibroma virus, rabbit fibroma virus, squirrel fibroma virus, or myxoma virus. In a preferred embodiment, the helper virus is an SFV. In some embodiments, the helper virus is an Orthopoxvirus. In some embodiments, the Orthopoxvirus is a camelpox virus (CMLV), cowpox virus (CPXV), ectromelia virus (ECTV, “mousepox agent”), HPXV, monkeypox virus (MPXV), rabbitpox virus (RPXV), raccoonpox virus, skunkpox virus, Taterapox virus, Uasin Gishu disease virus, VACV and volepox virus (VPV). In some embodiments, the helper virus is an Avipoxvirus, Capripoxvirus, Cervidpoxvirus, Crocodylipoxvirus, Molluscipoxvirus, Parapoxvirus, Suipoxvirus, or Yatapoxvirus. In some embodiments, the helper virus is a fowlpox virus. In some embodiments, the helper virus is an Alphaentomopoxvirus, Betaentomopoxvirus, or Gammaentomopoxvirus. In some embodiments, the helper virus is a psoralen-inactivated helper virus. In an exemplary embodiment of the disclosure, an scPV is reactivated from overlapping DNA fragments transfected into SFV-infected BGMK cells. The SFV is then eliminated by plating the mixed viral progeny on BSC-40 cells.

The skilled worker will understand that appropriate host cells to be used for the reactivation of the scPV and the selection and/or isolation of the scPV will depend on the particular combination of helper virus and chimeric poxvirus being produced by the methods of the invention. Any host cell that supports the growth of both the helper virus and the scPV may be used for the reactivation step and any host cell that does not support the growth of the helper virus may be used to eliminate the helper virus and select and/or isolate the scPV. In some embodiments, the helper virus is a Leporipoxvirus and the host cells used for the reactivation step may be selected from rabbit kidney cells (e.g., LLC-RK1, RK13, etc.), rabbit lung cells (e.g., R9ab), rabbit skin cells (e.g., SF1Ep, DRS, RAB-9), rabbit cornea cells (e.g., SIRC), rabbit carcinoma cells (e.g., Oc4T/cc), rabbit skin/carcinoma cells (e.g., CTPS), monkey cells (e.g., Vero, BGMK, etc.) or hamster cells (e.g., BHK-21, etc.). In some embodiments, the helper virus is SFV.

In various aspects, the scPVs of the present invention can be propagated in any substrate that allows the virus to grow to titers that permit the uses of the scPVs described herein. In one embodiment, the substrate allows the scPVs to grow to titers comparable to those determined for the corresponding wild-type viruses. In some embodiments, the scPVs may be grown in cells (e.g., avian cells, bat cells, bovine cells, camel cells, canary cells, cat cells, deer cells, equine cells, fowl cells, gerbil cells, goat cells, human cells, monkey cells, pig cells, rabbit cells, raccoon cells, seal cells, sheep cells, skunk cells, vole cells, etc.) that are susceptible to infection by the poxviruses. Such methods are well-known to those skilled in the art. Representative mammalian cells include, but are not limited to BHK, BGMK, BRL3A, BSC-40, CEF, CEK, CHO, COS, CVI, HaCaT, HEL, HeLa cells, HEK293, human bone osteosarcoma cell line 143B, MDCK, NIH/3T3, Vero cells, etc.). For virus isolation, the scPV is removed from cell culture and separated from cellular components, typically by well-known clarification procedures, e.g., such as gradient centrifugation and column chromatography, and may be further purified as desired using procedures well known to those skilled in the art, e.g., plaque assays.

### Pharmaceutical Composition of the Disclosure

In one aspect, the invention relates to a pharmaceutical composition comprising the isolated stem cell or population thereof of the invention and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition (e.g., immunogenic or vaccine formulation) is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In some embodiments, the pharmaceutical composition of the invention may be administered by standard routes of administration. Many methods may be used to introduce the formulations into a subject, these include, but are not limited to, intranasal, intratracheal, oral, intradermal, intramuscular, intraperitoneal, intravenous, conjunctival and subcutaneous routes.

### Exemplary Uses

Method for Delivering the scPV by the Stem Cells of the Disclosure

In one aspect, the invention relates to a method for delivery of a synthetic chimeric poxvirus (scPV) into a subject, the method comprising infecting the stem cells of the invention with a synthetic chimeric poxvirus and administering the scPV-infected stem cells into the subject.

Methods to infect the stem cells of the invention with a synthetic chimeric poxvirus are known in the art. Those skilled in the art can determine appropriate parameters, such as number of stem cells and multiplicity of infection.

In one embodiment, the stem cells are autologous and the invention relates to a method for delivery of a scPV into a subject, the method comprising (a) obtaining the stem cells from said subject; (b) infecting the stem cells with the oncolytic scPV and (c) administer the scPV infected stem cells back into the subject. In another embodiment, the stem cells are mesenchymal stem cells (MSC). In another embodiment, the MSC derive from the subject's adipose tissue.

In another embodiment, the stem cells are allogeneic and the invention relates to a method for delivery of a scPV into a subject, the method comprising (a) obtaining the stem cells from a subject; (b) infecting the stem cells with the oncolytic scPV and (c) administer the scPV infected stem cells back into the subject.

In some embodiments, the stem cells of the invention are administered in a single administration or in multiple administrations.

The stem cells can be implanted locally at the site of cell damage or dysfunction, or systemically. Exemplary routes of administration of the stem cell compositions include, but are not limited to, intravenous, intramuscular, intradermal, intraperitonal, intracoronary, intramyocardial, transendocardial, trans-epicardial, intraspinal, intra-arterial, intra-striatum, intratumoral, topical, transdermal, rectal or sub-epidermal routes. The most suitable route for administration will vary depending upon the disorder or condition to be treated, such as the location of cell damage or dysfunction. For example, stem cells can be administered intraarterially or intra-spinally at the site of injury for the treatment of spinal cord injury. In other examples, stem cell compositions can be administered by an intracoronary, intramyocardial, transendocardial or trans-epicardial route for the treatment of cardiovascular disease. In a preferred embodiment, the administration is intravenously.

In one embodiment, the amount of scPV used for the stem cells infection is 1×10⁵ or about 1×10⁵ plaque forming units (PFU), 5×10⁵ or about 5×10⁵ PFU, at least 1×10⁶ or about 1×10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU.

In one embodiment, the multiplicity of infection of scPV used for the stem cells infection is about 0.5 PFU/cell, or about 1 PFU/cell, or about 2 PFU/cell, or about 3 PFU/cell, or about 4 PFU/cell, or about 5 PFU/cell, or about 6 PFU/cell, or about 7 PFU/cell, or about 8 PFU/cell, or about 9 PFU/cell or about 10 PFU/cell.

### Conditions Susceptible to be Treated by the Exemplary Stem Cells of the Disclosure

Conditions amenable to stem cell therapy include any in which one or more cell populations are defective or have been depleted or destroyed. Such conditions include degenerative disorders or conditions and acute or chronic injuries. Once the stem cells are implanted or engrafted into the patient, such as at the location of cell or tissue damage or dysfunction, the stem cells can differentiate into the desired cell or tissue type based on the physical and chemical signals in the local extracellular microenvironment, thereby replacing the destroyed or defective cells with functional healthy cells. Exemplary conditions include, but are not limited to, cancer, cardiovascular disease, diabetes, spinal cord injury, neurodegenerative disease, traumatic brain injury, Alzheimer's disease, Parkinson's disease, multiple sclerosis (MS), Amyotrophic lateral sclerosis (ALS), Duchenne Muscular Dystrophy, muscle damage or dystrophy, stroke, burns, lung disease, retinal disease, kidney disease, osteoarthritis, and rheumatoid arthritis.

### Method of Treating Cancer

As used herein, a method for treating or preventing cancer means that any of the symptoms, such as the tumor, metastasis thereof, the vascularization of the tumors or other parameters by which the disease is characterized are reduced, ameliorated, prevented, placed in a state of remission, or maintained in a state of remission. It also means that the indications of cancer and metastasis can be eliminated, reduced or prevented by the treatment. Non-limiting examples of the indications include uncontrolled degradation of the basement membrane and proximal extracellular matrix, migration, division, and organization of the endothelial cells into new functioning capillaries, and the persistence of such functioning capillaries.

The synthetic chimeric poxviruses (scPVs) of the various aspects of the invention can be used as oncolytic agents that selectively replicate in and kill cancer cells. Cells that are dividing rapidly, such as cancer cells, are generally more permissive for poxviral infection than non-dividing cells. Many features of poxviruses, such as safety in humans, ease of production of high-titer stocks, stability of viral preparations, and capacity to induce antitumor immunity following replication in tumor cells make poxviruses desirable oncolytic agents. The scPVs produced according to the methods of the invention may comprise one or modifications that render them suitable for the treatment of cancer. Accordingly, in one aspect, the disclosure provides a method of inducing death in cancer cells, the method comprising contacting the cancer cells with an isolated scPV or pharmaceutical composition comprising an scPV of the invention or the stem cells of the invention. In one aspect, the disclosure provides a method of treating cancer, the method comprising administering to a patient in need thereof, a therapeutically effective amount of the stem cells of the invention or the pharmaceutical composition of the invention. Another aspect includes the use of the stem cells of the invention or the pharmaceutical composition described herein to induce death in a neoplastic disorder cell such as a cancer cell or to treat a neoplastic disorder such as cancer. In some embodiments, the poxvirus oncolytic therapy is administered in combination with one or more conventional cancer therapies (e.g., surgery, chemotherapy, radiotherapy, thermotherapy, and biological/immunological therapy). In specific embodiments, the oncolytic virus is a synthetic chimeric VACV (scVACV) of this invention. In some embodiments, the oncolytic virus is a synthetic chimeric myxoma virus of this invention. In some embodiments, the oncolytic virus is a synthetic chimeric HPXV (scHIPXV) of this invention. In some embodiments, the oncolytic virus is a synthetic chimeric raccoonpox virus of this invention. In some embodiments, the oncolytic virus is a synthetic chimeric yaba-like disease virus of this invention.

In various aspects, using the method of treatment of cancer of this invention, one or more desirable genes can be easily introduced, and one or more undesirable genes can be easily deleted from the synthetic chimeric poxviral genome. In some embodiments, the scPVs of the invention for use as oncolytic agents are designed to express transgenes to enhance their immunoreactivity, antitumor targeting and/or potency, cell-to-cell spread and/or cancer specificity. In some embodiments, an scPV of the invention is designed or engineered to express an immunomodulatory gene (e.g., GM-CSF, or a viral gene that blocks TNF function). In some embodiments, an scPV of the disclosure is designed to include a gene that expresses a factor that attenuates virulence. In some embodiments, an scPV of the invention is designed or engineered to express a therapeutic agent (e.g., hEPO, BMP-4, antibodies to specific tumor antigens or portions thereof, etc.). In some embodiments, the scPVs of the invention have been modified for attenuation. In some embodiments, the scPV of the invention is designed or engineered to lack the viral TK gene. In some embodiments, an scVACV of the invention is designed or engineered to lack vaccinia growth factor gene. In some embodiments, an scVACV of the invention is designed or engineered to lack the hemagglutinin gene.

The stem cells of the various embodiments of the invention are useful for treating a variety of neoplastic disorders and/or cancers. In some embodiments, the type of cancer includes but is not limited to bone cancer, breast cancer, bladder cancer, cervical cancer, colorectal cancer, esophageal cancer, gliomas, gastric cancer, gastrointestinal cancer, head and neck cancer, hepatic cancer such as hepatocellular carcinoma, leukemia, lung cancer, lymphomas, ovarian cancer, pancreatic cancer, prostate cancer, renal cancer, skin cancer such as melanoma, testicular cancer, etc. or any other tumors or pre-neoplastic lesions that may be treated.

In another embodiment, the method further comprises detecting the presence of the administered scPV, in the neoplastic disorder or cancer cell and/or in a sample from a subject administered an isolated or recombinant virus or composition described herein. For example, the subject can be tested prior to administration and/or following administration of the scPV or composition described herein to assess for example the progression of the infection. In some embodiments, an scPV of the disclosure comprises a detection cassette and detecting the presence of the administered chimeric poxvirus comprises detecting the detection cassette encoded protein. For example, wherein the detection cassette encodes a fluorescent protein, the subject or sample is imaged using a method for visualizing fluorescence.

In some embodiments, the stem cells of the various embodiments of the invention are administered in a single administration or in multiple administrations.

In other embodiments, the stem cells of the invention can be implanted locally at the site of cell damage or dysfunction, or systemically in order to be used in a method to treat cancer. Exemplary routes of administration of the stem cell compositions include, but are not limited to, intravenous, intramuscular, intradermal, intraperitonal, intracoronary, intramyocardial, transendocardial, trans-epicardial, intraspinal, intra-arterial, intra-striatum, intratumoral, topical, transdermal, rectal or sub-epidermal routes. The most suitable route for administration will vary depending upon the disorder or condition to be treated, such as the location of cell damage or dysfunction. For example, stem cells can be administered intraarterially or intra-spinally at the site of injury for the treatment of spinal cord injury. In other examples, stem cell compositions can be administered by an intracoronary, intramyocardial, transendocardial or trans-epicardial route for the treatment of cardiovascular disease. In a preferred embodiment, the administration is intravenously.

The scPV for use in a method of treatment of cancer can encode a therapeutic gene product. In some examples, the therapeutic gene product is an anti-cancer agent or anti-angiogenic agent. In one embodiment, the therapeutic gene product can be selected from among a cytokine, a chemokine, an immunomodulatory molecule, an antigen, an antibody or fragment thereof, an antisense RNA, a prodrug converting enzyme, an siRNA, an angiogenesis inhibitor, a toxin, an antitumor oligopeptide, a mitosis inhibitor protein, an antimitotic oligopeptide, an anti-cancer polypeptide antibiotic, a transporter protein, and a tissue factor.

The method for treatment of cancer including the stem cells of the invention can also include administering an anticancer agent. Exemplary anticancer agents include, but are not limited to, a cytokine, a chemokine, a growth factor, a photosensitizing agent, a toxin, an anti-cancer antibiotic, a chemotherapeutic compound, a radionuclide, an angiogenesis inhibitor, a signaling modulator, an antimetabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, hyperthermia or hyperthermia therapy, a bacterium, radiation therapy and a combination of such agents. In some examples, the anticancer agent is cisplatin, carboplatin, gemcitabine, irinotecan, an anti-EGFR antibody, or an anti-VEGF antibody. In some examples, the anticancer agent is administered simultaneously, sequentially, or intermittently with the virus.

In methods provided herein for administering the oncolytic virus to a subject, the method can also include administering an anti-viral agent to attenuate replication of or eliminate the virus from the subject during or following therapy. Exemplary antiviral agents include, but are not limited to, cidofovir, alkoxyalkyl esters of cidofovir, Gleevec, gancyclovir, acyclovir and ST-26.

In one aspect of the method for treatment of cancer, the scPV contains a gene deletion. A gene deletion is understood to be the loss or absence of a DNA sequence of a gene, or a deficiency in that gene or a deletion mutation, in which part of a chromosome or a sequence of DNA is lost during DNA replication. In one embodiment, the deleted gene is selected from a gene encoding a protein or fragment thereof, a gene segment that regulates transcription, a gene segment that regulates viral replication, a gene segment that affects cellular mitosis, a gene segment that affects cellular metabolism, a gene segment that encodes an antisense RNA, a gene segment that encodes an siRNA, a gene segment that regulates angiogenesis, a gene segment that regulates one or more transporter proteins, or a gene segment that regulates one or more tissue factors. In another embodiment, the gene deletion potentiates the anti-cancer or the anti-angiogenic effect of the virus. The term “potentiates the effect”, as used herein, means that the anti-cancer or the anti-angiogenic compounds are more effective or more active after the gene deletion, which means that the activity of the compounds is augmented after the gene deletion was performed on the scPV.

### Method of Treating a Variola Virus Infection

The stem cells of the various aspects of the invention can be used to treat a variola virus infection. With respect to infections (e.g., a poxviral infection or a variola virus infection), treatment refers to the eradication or control of the replication of an infectious agent (e.g., the poxvirus or the variola virus), the reduction in the numbers of an infectious agent (e.g., the reduction in the titer of the virus), the reduction or amelioration of the progression, severity, and/or duration of an infection (e.g., a poxviral/variola infection or a condition or symptoms associated therewith), or the amelioration of one or more symptoms resulting from the administration of one or more therapies (including, but not limited to, the administration of one or more prophylactic or therapeutic agents).

In some embodiments, the stem cells of the invention are administered in a single administration or in multiple administrations for the treatment of a variola infection.

Exemplary routes of administration of the stem cell compositions include, but are not limited to, intravenous, intramuscular, intradermal, intraperitonal, intracoronary, intramyocardial, transendocardial, trans-epicardial, intraspinal, intra-arterial, intra-striatum, intratumoral, topical, transdermal, rectal or sub-epidermal routes.

### EXAMPLES

### Example 1. Synthetic Chimeric HPXV (scHPXV)

### Selection and Design of Overlapping Fragments of the Viral Genome

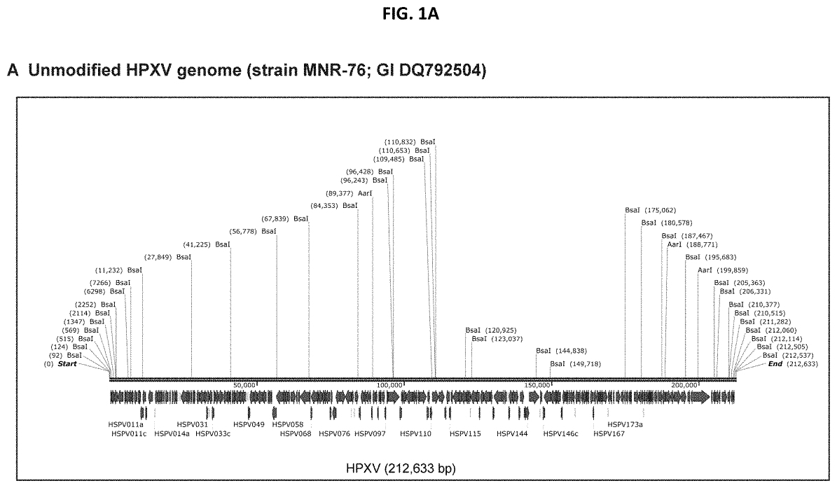

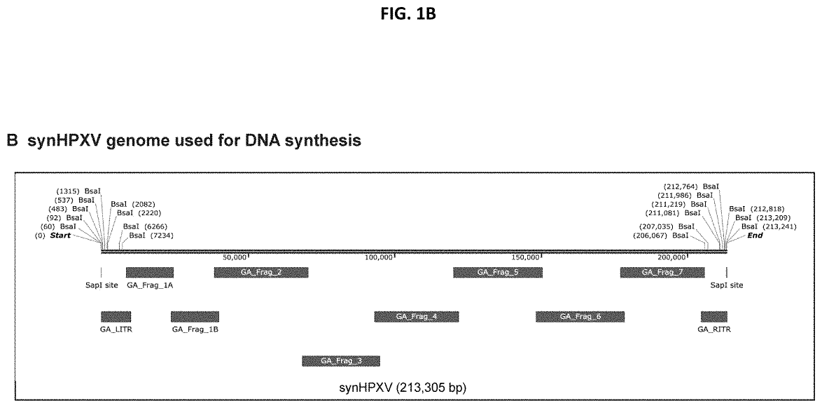

Design of the scHPXV genome was based on the previously described genome sequence for HPXV (strain MNR-76; FIG. 1A) [GenBank accession DQ792504] (Tulman E R, Delhon G, Afonso C L, Lu Z, Zsak L, Sandybaev N T, et al. Genome of horsepox virus. Journal of Virology. 2006; 80(18):9244-58). The 212,633 bp genome was divided into 10 overlapping fragments (FIG. 1B). These fragments were designed so that they shared at least 1.0 kbp of overlapping sequence (i.e. homology) with each adjacent fragment, to provide sites where homologous recombination will drive the assembly of full-length genomes (Table 1). These overlapping sequences will provide sufficient homology to accurately carry out recombination between the co-transfected fragments (Yao X D, Evans D H. High-frequency genetic recombination and reactivation of orthopoxviruses from DNA fragments transfected into leporipoxvirus-infected cells. Journal of Virology. 2003; 77(13):7281-90). The terminal 40 bp from the HPXV genome sequence (5′-TTTATTAAATTTTACTATTTATTTAGTGTCTAGAAAAAAA-3′) (SEQ ID NO: 50) was not included in the synthesized inverted terminal repeat (ITR) fragments. Instead, a SapI restriction site was added at the 5′-terminus (GA_LITR) and 3′-terminus (GA_RITR) of the ITR fragments followed by a TGT sequence. These SapI restriction sites were used to ligate the VACV terminal hairpins onto the ITR fragments (described below).

Each fragment was chemically synthesized and subcloned into a plasmid using terminal SfiI restriction sites on each fragment. To assist with sub-cloning these fragments, AarI and BsaI restriction sites were silently mutated in all the fragments, except for the two ITR-encoding fragments (Table 2). The BsaI restriction sites in the two ITR-encoding fragments were not mutated, in case these regions contained nucleotide sequence-specific recognition sites that were important for efficient DNA replication and concatemer resolution.

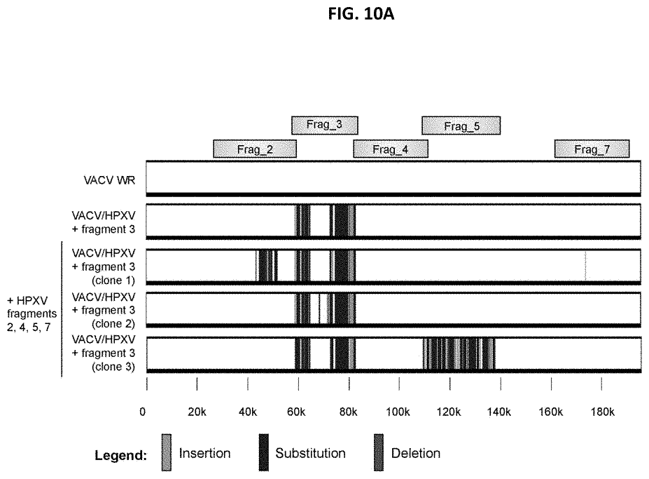

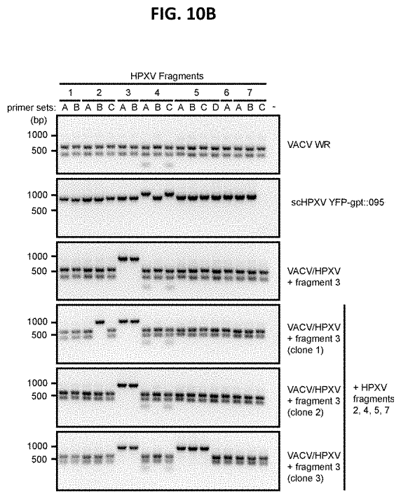

A yfp/gpt cassette under the control of a poxvirus early late promoter was introduced into the HPXVO95/J2R locus within GA_Fragment_3) so that reactivation of HPXV (scHPXV YFP-gpt::095) was easy to visualize under a fluorescence microscope. The gpt locus also provided a potential tool for selecting reactivated viruses using drug selection. HPXV095 encodes the HPXV homolog of the non-essential VACV J2R gene and by co-transfecting Fragment_3 and other HPXV clones into SFV-infected BGMK cells, along with VACV DNA, a variety of hybrid viruses were recovered, validating the selection strategy (FIGS. 10A and 10B). Silent mutations were also introduced into the HPXV044 (VACV^{WR}F4L) sequence (GA_Fragment_2) to create two unique restrictions sites within GA_Fragment_2 (Table 3). In some embodiments, these unique restriction sites may be used to rapidly introduce recombinant gene products (such as but not limited to, selectable markers, fluorescent proteins, antigens, etc.) into GA_Fragment_2 prior to reactivation of HPXV.

TABLE 1
The HPXV genome fragments used in this study. The size of each
fragment and location within the HPXV genome are indicated.

| | | Location within |
|---|---|---|
| | | HPXV [DQ792504] |
| Fragment Name | Size (bp) | (bp) |
| GA_Left ITR (SEQ ID NO: 1) | 10,095 | 41-10,135 |
| GA_Fragment 1A (SEQ ID NO: 2) | 16,257 | 8505-24,761 |
| GA_Fragment 1B (SEQ ID NO: 3) | 16,287 | 23764-40,050 |
| GA_Fragment 2 (SEQ ID NO: 4) | 31,946 | 38,705-70,650 |
| GA_Fragment 3 (SEQ ID NO: 5) | 25,566 | 68,608-94,173 |
| GA_Fragment 4 (SEQ ID NO: 6) | 28,662 | 92,587-121,248 |
| GA_Fragment 5 (SEQ ID NO: 7) | 30,252 | 119,577-149,828 |
| GA_Fragment 6 (SEQ ID NO: 8) | 30,000 | 147,651-177,650 |
| GA_Fragment 7 (SEQ ID NO: 9) | 28,754 | 176,412-205,165 |
| GA_Right ITR (SEQ ID NO: 10) | 8,484 | 204,110-212,593 |

TABLE 2
Silent mutations created in scHPXV YFP-gpt::095 fragments
to remove AarI and BsaI restriction sites from HPXV genome.

| | | Nucleotide | | | |
|---|---|---|---|---|---|
| | | change in | | | Mutation |
| | Restriction | coding | | Location | verified by |
| | endonuclease | strand of | | in HPXV | whole |
| GA_HPXV | recognition | HPXV | HPXV | genome | genome |
| Fragment | site removed | genome | Gene | [DQ792504] | sequencing |
| GA_Frag_1A | BsaI | A to G | HPXV011a | 11,228 | ✓ |
| GA_Frag_1B | BsaI | A to G | HPXV025 | 27,845 | ✓ |
| GA_Frag_2 | BsaI | A to G | HPXV040 | 41,232 | ✓ |
| | BsaI | G to A | HPXV059 | 56,775 | ✓ |
| | BsaI | G to A | HPXV066 | 67,836 | ✓ |
| GA_Frag_3 | BsaI | G to A | HPXV083 | 84,361 | ✓ |
| | AarI | T to C | HPXV091 | 89,368 | ✓ |
| GA_Frag_4 | BsaI | T to C | HPXV099 | 96,239 | ✓ |
| | BsaI | A to G | HPXV099 | 96,437 | ✓ |
| | BsaI | A to G | HPXV110 | 109,492 | ✓ |
| | BsaI | A to G | HPXV111 | 110,661 | ✓ |
| | BsaI | G to A | HPXV111 | 110,840 | ✓ |
| GA_Frag_4 | BsaI | C to T | HPXV119 | 120,933 | ✓ |
| GA_Frag_5 | | | | | |
| GA_Frag_5 | BsaI | A to G | HPXV123 | 123,035 | ✓ |
| | BsaI | T to C | HPXV145 | 144,834 | ✓ |
| GA_Frag_5 | BsaI | T to C | HPXV146d | 149,727 | ✓ |
| GA_Frag_6 | | | | | |
| GA_Frag_6 | BsaI | G to A | HPXV178b | 175,070 | ✓ |
| GA_Frag_7 | BsaI | G to A | HPXV182 | 180,573 | ✓ |
| | BsaI | A to G | HPXV192 | 187,476 | ✓ |
| | AarI | G to A | HPXV193 | 188,761 | ✓ |
| | BsaI | C to T | HPXV197 | 195,680 | ✓ |
| | AarI | T to C | HPXV200 | 199,873 | ✓ |

TABLE 3
Introduction of silent nucleotide mutations in the HPXV044
(VACV F4L) gene to create unique restriction endonuclease sites in
GA_Fragment_2.

| | Restriction | Nucleotide change | |
|---|---|---|---|
| | endonuclease | in the HPXV | Location in |
| HPXV gene | site created | coding strand | HPXV genome |
| HPXV044 | AvaI | A to C | 44,512 |
| | StuI | A to C | 45,061 |

Synthesis of the Slow (S) and Fast (F) forms of the terminal hairpin loops from VACV (strain WR)

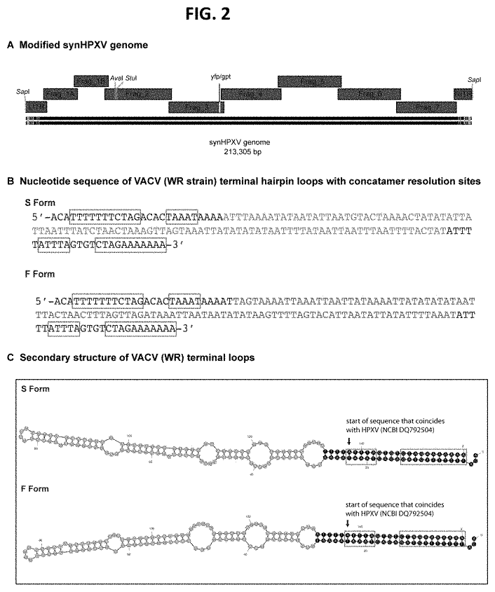

The Slow (S) and Fast (F) forms of the terminal hairpin loops from VACV (strain WR) were synthesized as 157 nt ssDNA fragments (Integrated DNA Technologies; FIG. 2B). Through DNA synthesis, a 5′ overhang comprised of three nucleotides was left at the end of each hairpin (5′-ACA; FIG. 2C). The concatemer resolution site from the HPXV sequence [DQ792504] was also synthesized in the terminal hairpin loops (FIG. 2B).

Digestion and Purification of scHPXV YFP-Gpt::095 Fragments

Synthetic HPXV fragments were digested with SfiI overnight at 50° C. The scHPXV ITR fragments were individually digested with SapI (ThermoFisher Scientific) for 1 h, inactivated at 65° C. for 10 minutes, before digestion with SfiI overnight at 50° C. Approximately 1 U of FastAP alkaline phosphatase was added to the scHPXV YFP-gpt::095 ITR digestions and incubated at 37° C. for an additional 1 h. All scHPXV YFP-gpt::095 fragments were subsequently purified using a QiaexII DNA cleanup kit (Qiagen). All scHPXV YFP-gpt::095 fragments were eluted from the QiaexII suspension in 10 mM Tris-HCl. DNA concentrations were estimated using a NanoDrop (ThermoFisher Scientific).

Poxviruses catalyze very high-frequency homologous recombination reactions that are inextricably linked to the process of virus replication. Herein, it is demonstrated that large fragments of chemically synthesized HPXV duplex DNA can be joined to form a functional scHPXV genome using virus-catalyzed recombination and replication reactions.

Using the published sequence of the HPXV genome (strain WNR-76), the 212,633 bp genome was divided into 10-overlapping fragments (FIG. 2). All of the BsaI and AarI sites in every fragment except the ITRs were mutated, in case sequence-specific sites within this region are unknowingly required for efficient genome replication and concatemer resolution. As described above, to facilitate the addition of the terminal hairpin loop structures from VACV onto the end of the ITRs, a SapI recognition site was included next to the left- and right-terminal end of both LITR and RITR fragments, respectively (FIG. 2A). These SapI sites were embedded within the flanking vector sequences, and the SapI enzyme cuts downstream of the site, outside of the recognition sequence and in the HPXV DNA. Thus, when DNA was cut with SapI, it left sticky ends within the DNA copied from the HPXV sequence and thus permitted the assembly of a precise sequence copy (through a subsequent ligation), containing no extraneous restriction sites. The other ends of the LITR and RITR fragments (the internal ends with respect to the genome map) were each bounded by SfiI recognition sites, as were both ends of the remaining HPXV fragments. All of these DNAs were supplied in a plasmid form for easy propagation. To prepare the internal fragments for transfection into SFV-infected cells, these plasmids were digested with SfiI to release the plasmid from each scHPXV YFP-gpt::095 fragment (see below for how the LITR and RITR fragments are processed). Following digestion, each reaction was purified to remove any contaminating enzyme, but the plasmid was not removed from the digestion and was co-transfected alongside each scHPXV YFP-gpt::095 fragment. This did not interfere with the reaction and was done to minimize the amount of DNA manipulation and possible fragmentation of these large DNA fragments.

While the reaction efficiency may be affected by the number of transfected fragments, greater than or less than 10 overlapping fragments may be used in the methods of the disclosure. Without being bound by theory, ˜15 fragments may represent a practical upper limit without further optimization of the reactivation reaction. The ideal lower limit would be a single genome fragment, but in practice the telomeres are most easily manipulated as more modest-sized fragments (e.g., ˜10 kb).

Ligation of VACV F- and S-Terminal Hairpin Loops onto scHPXV YFP-Gpt::095 Left and Right ITR Fragments

Approximately one microgram of each of the terminal VACV hairpin loops was incubated at 95° C. for 5 minutes followed by a “snap” cool on ice to form the hairpin structure. The hairpin loops were subsequently phosphorylated at their 5′ end before ligation. Briefly, separate 20 μl reactions containing 1 μg of either VACV F-hairpin or VACV S-hairpin, 2 μl of 10×T4 polynucleotide kinase buffer (ThermoFisher Scientific), 1 mM ATP, and 10 units of T4 polynucleotide kinase (ThermoFisher Scientific) were incubated at 37° C. for 1 h. The reaction was terminated by heat inactivation at 75° C.

Approximately one microgram of either left ITR or right ITR was incubated separately with a 20-fold molar excess of each terminal hairpin in the presence of 5% PEG-4000, and 5 units of T4 DNA ligase overnight at 16° C. Each ligation reaction was heat-inactivated at 65° C. for 10 minutes followed by incubation on ice until ready to transfect into cells.

Orthopoxviruses encode linear dsDNA genomes bearing variable length inverted terminal repeats (ITR) at each end of the genome. The two strands of the duplex genome are connected by hairpin loops to form a covalently continuous polynucleotide chain. The loops are A+T-rich, cannot form a completely base-paired structure, and exist in two forms that are inverted and complementary in sequence (Baroudy B M, Venkatesan S, Moss B). Incompletely base-paired flip-flop terminal loops link the two DNA strands of the vaccinia virus genome into one uninterrupted polynucleotide chain. Cell. 1982; 28(2):315-24) (FIG. 2B). They are called slow [S] and fast [F] forms based upon their electrophoretic properties and probably fold into partially duplex hairpin structures that cap the ends of the linear dsDNA genome (FIG. 2C). The published sequence of the HPXV genome was incomplete, probably missing ˜60 bp from the terminal ends, making it impossible to precisely replicate the HPXV hairpins. Instead, 157 nt ssDNA fragments were chemically synthesized using the published sequence of the VACV telomeres as a guide and leaving a 5′ overhang comprised of three nucleotides at the end of each hairpin (5′-ACA; FIG. 2C) (Baroudy B M, Venkatesan S, Moss B). Incompletely base-paired flip-flop terminal loops link the two DNA strands of the vaccinia virus genome into one uninterrupted polynucleotide chain. Cell. 1982; 28(2):315-24). This overhang is complementary to the ends generated by cutting cloned LITR and RITR fragments with SapI.

Sequences derived from VACV were used based upon data suggesting a close common ancestry between HPXV and VACV. It may be possible to use other terminal hairpins from other poxviruses since there are sequence features that are commonly conserved between the hairpin ends of different Chordopoxviruses. For example, the resolution sites in the hairpin ends are highly conserved in both sequence and functionality (they resemble late promoters).

These single-stranded oligonucleotides were heated to 95° C. and then quickly chilled on ice to form the incompletely base-paired terminal hairpin (FIG. 2C). Next, each oligonucleotide was phosphorylated and ligated separately at 20-fold molar excess with either the left or right ITR fragment previously digested with both SapI and SfiI. Digestion of the ITRs with these enzymes resulted in a 5′-TGT overhang at the 5′ termini of each ITR, which was complementary to the 5′-ACA overhang in the terminal hairpin loop structure. This produced a hairpin-terminated copy of each ITR.

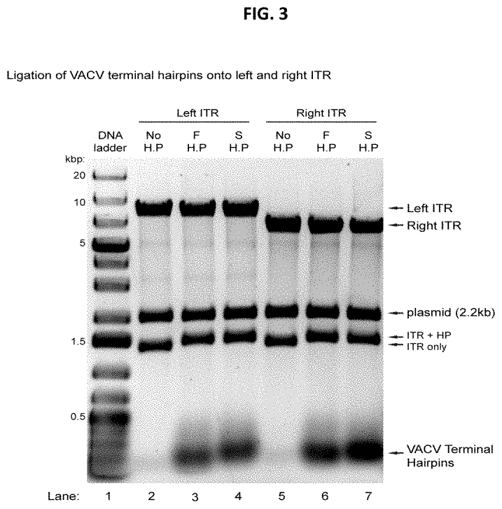

To confirm that a hairpin-terminated structure was added to both ITR fragments, restriction digestion of the ITR fragments with PvuII was performed. Since it was impossible to visualize the addition of a ˜70 bp terminal hairpin onto the terminus of a ˜10 kb ITR by gel electrophoresis, a small amount of each ligation was digested with PvuII. If no terminal hairpin was ligated to the ITR, then digestion with PvuII resulted in a 1472 bp product (FIG. 3, lanes 2 and 5). If, however, the terminal hairpin loop was successfully added to the HPXV ITRs, then an increase in the size of the ITR fragment was seen on an agarose gel (FIG. 3, compare lane 2 with 3 and 4; compare lane 5 with 6 and 7). These data suggested that under these conditions almost all of the HPXV ITRs contained terminal hairpin loops at one end of the fragment.

Reactivation of scHPXV YFP-Gpt:: 095 from Chemically Synthesized dsDNA Fragments

SFV strain Kasza and BSC-40 were originally obtained from the American Type Culture Collection. Buffalo green monkey kidney (BGMK) cells were obtained from G. McFadden (University of Florida). BSC-40 and BGMK cells are propagated at 37° C. in 5% CO₂ in minimal essential medium (MEM) supplemented with L-glutamine, nonessential amino acids, sodium pyruvate, antibiotics and antimycotics, and 5% fetal calf serum (FCS; ThermoFisher Scientific).

Buffalo green monkey kidney (BGMK) cells were grown in MEM containing 60 mm tissue-culture dishes until they reached approximately 80% confluency. Cells were infected with Shope Fibroma Virus (SFV) in serum-free MEM at a MOI of 0.5 for 1 h at 37° C. The inoculum was replaced with 3 ml of warmed MEM containing 5% FCS and returned to the incubator for an additional hour. Meanwhile, transfection reactions were set up as follows. Lipofectamine complexes were prepared by mixing approximately 5 μg total synthetic HPXV DNA fragments in 1 ml Opti-MEM with Lipofectamine2000 diluted in 1 ml Opti-MEM at a ratio of 3:1 (Lipofectamine2000 to total DNA). The complexes were incubated at room temperature for 10 minutes and then added dropwise to the BGMK cells previously infected with SFV. Approximately 16 h post infection, the media was replaced with fresh MEM containing 5% FCS. The cells were cultured for an additional 4 d (total of 5 d) at 37° C. Virus particles were recovered by scraping the infected cells into the cell culture medium and performing three cycles of freezing and thawing. The crude extract was diluted 10² in serum-free MEM and 4 ml of the inoculum was plated on 9-16 150 mm tissue culture plates of BSC-40 cells to recover reactivated scHPXV YFP-gpt::095. One hour post infection, the inoculum was replaced with MEM containing 5% FCS and 0.9% Noble Agar. Yellow fluorescent plaques were visualized under an inverted microscope and individual plaques were picked for further analysis. ScHPXV YFP-gpt::095 plaques were plaque purified three times with yellow fluorescence selection.

SFV-catalyzed recombination and reactivation of Orthopoxvirus DNA to assemble recombinant vaccinia viruses had previously been described (Yao X D, Evans D H. High-frequency genetic recombination and reactivation of orthopoxviruses from DNA fragments transfected into leporipoxvirus-infected cells. Journal of Virology. 2003; 77(13):7281-90). Construction of recombinant vaccinia viruses using leporipoxvirus-catalyzed recombination and reactivation of orthopoxvirus DNA. Methods Mol Biol. 2004; 269:51-64). Several biological features make this an attractive model system. First, SFV has a narrow host range, productively infecting rabbit cells and certain monkey cell lines, like BGMK. It can infect, but grows very poorly on cells like BSC-40. Second, it grows more slowly compared to Orthopoxviruses, taking approximately 4-5 days to form transformed “foci” in monolayers of cells, a characteristic that is very different from Orthopoxviruses, which produce plaques within 1-2 days in culture. This difference in growth between Leporipoxviruses and Orthopoxviruses allows one to differentiate these viruses by performing the reactivation assays in BGMK cells and plating the progeny on BSC-40 cells. In some embodiments, other helper viruses (such as but not limited to fowlpox virus) may be used. In some embodiments, different cell combinations may be used.

BGMK cells were infected with SFV at a MOI of 0.5 and then transfected with 5 μg of digested GA_HPXV fragments 2 h later. Five days post transfection all of the infectious particles were recovered by cell lysis and re-plated on BSC-40 cells, which only efficiently supported growth of HPXV (or other Orthopoxviruses). The resulting reactivated scHPXV YFP-gpt::095 plaques were visualized under a fluorescence microscope. The visualization was enabled by the yfp/gpt selectable marker in the HPXV095/J2R locus within Frag_3 (FIG. 2A). Virus plaques were detected in BSC-40 monolayers within 48 h of transfection. The efficiency of recovering scHPXV YFP-gpt::095 was dependent on a number of factors, including DNA transfection efficiency, but ranged up to a few PFU/μg of DNA transfected.

Confirmation of scHPXV YFP-Gpt::095 Genome Sequence by PCR and Restriction Fragment Analysis
PCR and Restriction Digestion Analysis of scHPXV

To rapidly confirm the presence of scHPXV YFP-gpt::095 in reactivated plaque picks, PCR primers were designed to flank individual BsaI sites that were mutated in the scHPXV (Table 5). Genomic scHPXV YFP-gpt::095 DNA was isolated from BSC-40 cells infected with scHPXV YFP-gpt::095 and used as a template. Genomic DNA from VACV-infected BSC-40 cells was used as a control to confirm the presence of BsaI sites within each PCR product. Following PCR amplification, reactions were subsequently digested with BsaI for 1 h at 37° C. PCR reactions were separated on a 1% agarose gel containing SYBR® safe stain to visualize DNA bands.

Further analysis of scHPXV YFP-gpt::095 genomes by restriction digestion followed by pulse-field gel electrophoresis (PFGE) was carried out on genomic DNA isolated using sucrose gradient purification (Yao X D, Evans D H. Construction of recombinant vaccinia viruses using leporipoxvirus-catalyzed recombination and reactivation of orthopoxvirus DNA. Methods Mol Biol. 2004; 269:51-64). Briefly, 100 ng of purified viral genomic DNA was digested with 5 U of BsaI or HindIII for 2 h at 37° C. Digested DNA was run on a 1% Seakem Gold agarose gel cast and run in 0.5× tris-borate-EDTA electrophoresis (TBE) buffer [110 mM tris; 90 mM borate; 2.5 mM EDTA]. The DNA was resolved on a CHEF DR-III apparatus (BioRad) at 5.7V/cm for 9.5 h at 14° C., using a switching time gradient of 1 to 10 s, a linear ramping factor, and a 1200 angle. This program allows resolution of DNA species from 1 kbp to >200 kbp. To resolve fragments from 75 bp to 5 kbp, electrophoresis on 1.5% agarose gel cast and run in 1.0×TBE at 115V for 2 h at room temperature was carried out. The DNA was visualized with SYBR® gold stain. The size of digested scHPXV YFP-gpt::095 DNA fragments was compared to control VACV genomic DNA.

TABLE 5
Primers that were used in this study to amplify
regions within VACV and HPXV surrounding the BsaI
restriction sites found in GA_Fragment_1A,
GA_Fragment_1B, GA_Fragment_2, GA_Fragment_3,
GA_Fragment_4, GA_Fragment_5, GA_Fragment_6, and
GA_Fragment_7.

| | | Position | Position |
|---|---|---|---|
| | | of BsaI | of BsaI |
| | Primer | site in | site in |
| | sequence | VACV | HPXV |
| Primer Name | (5′ to 3′) | [NC_006998] | [DQ792504] |
| HPXV 1A-FWD | CTGTATACCCATACT | 16,756 | 27,849 |
| (SEQ ID NO: 13) | GAATTGATGAAC | | |
| HPXV 1A-REV | GAGTTAATATAGACG | | |
| (SEQ ID NO: 14) | ACTTTACTAAAGTCA | | |
| | TG | | |
| HPXV 1B-FWD | GGTTCTTTTTATTCT | 23,076 | N/A |
| (SEQ ID NO: 15) | TTTAAACAGATCAAT | | |
| | GG | | |
| HPXV 1B-REV | TTCTTATTAAGACAT | | |
| (SEQ ID NO: 16) | TGAGCCCAGC | | |
| HPXV 2A-FWD | AGTCATCAATCATCA | 30,073 | 41,225 |
| (SEQ ID NO: 17) | TTTTTTCACC | | |
| HPXV 2A-REV | ATATAACGGACATTT | | |
| (SEQ ID NO: 18) | CACCACC | | |
| HPXV 2B-FWD | GTAACATATACAACT | 45,485 | 56,778 |
| (SEQ ID NO: 19) | TTTATTATGGCGTC | | |
| HPXV 2B-REV | CTAATCCACAAAAAA | | |
| (SEQ ID NO: 20) | TAGAATGTTTAGTTA | | |
| | TTTTG | | |
| HPXV 2C-FWD | AGTGACTGTATCCTC | 56,576 | 67,839 |
| (SEQ ID NO: 21) | AAACATCC | | |
| HPXV 2C-REV | TTTATAAAGGGTTAA | | |
| (SEQ ID NO: 22) | CCTTTGTCACATC | | |
| HPXV 3A-FWD | TTGTGTAGCGCTTCT | 60,981 | N/A |
| (SEQ ID NO: 23) | TTTTAGTC | | |
| HPXV 3A-REV | AAACGGATCCATGGT | | |
| (SEQ ID NO: 24) | AGAATATG | | |
| HPXV 3B-FWD | TATTTGCATCTGCTG | 84,916 | 84,353 |
| (SEQ ID NO: 25) | ATAATCATCC | | |
| HPXV 3B-REV | CGATGGATTCAAATG | | |
| (SEQ ID NO: 26) | ACTTGTTAATG | | |
| HPXV 4A-FWD | ATGCCTTTACAGTGG | 85,101 | 96,243 & |
| (SEQ ID NO: 27) | ATAAAGTTAAAC | | 96,428 |
| HPXV 4A-REV | CTGGATCCTTAGAGT | | |
| (SEQ ID NO: 28) | CTGGAAG | | |
| HPXV 4B-FWD | CGGAAAATGAAAAGG | 98,134 | 109,485 |
| (SEQ ID NO: 29) | TACTAGATACG | | |
| HPXV 4B-REV | TGAATAGCCGTTAAA | | |
| (SEQ ID NO: 30) | TAATCTATTTCGTC | | |
| HPXV 4C-FWD | TATGGATACATTGAT | 99,302 & | 110,653 & |
| (SEQ ID NO: 31) | AGCTATGAAACG | 99,481 | 110,832 |
| HPXV 4C-REV | AATACATCTGTTAAA | | |
| (SEQ ID NO: 32) | ATTGTTTGACCCG | | |
| HPXV 5A-FWD | CATTTTATTTCTAGA | 111,686 | 123,037 |
| (SEQ ID NO: 33) | CGTTGCCAG | | |
| HPXV 5A-REV | CGATATGAAACTTCA | | |
| (SEQ ID NO: 34) | GGCGG | | |
| HPXV 5B-FWD | ACAAAACGATTTAAT | 122,484 | N/A |
| (SEQ ID NO: 35) | TACAGAGTTTTCAG | | |
| HPXV 5B-REV | GTCCGGTATGAGACG | | |
| (SEQ ID NO: 36) | ACAG | | |
| HPXV 5C-FWD | TTAGGGATCACATGA | 133,505 | 144,838 |
| (SEQ ID NO: 37) | ATGAAATTCG | | |
| HPXV 5C-REV | TATGGAAGTTCCGTT | | |
| (SEQ ID NO: 38) | TCATCCG | | |
| HPXV 5D-FWD | GACTTGATAATCATA | 138,306 | 149,718 |
| (SEQ ID NO: 39) | TATTAAACACATTGG | | |
| | ATC | | |
| HPXV 5D-REV | AGATCTCCAGATTTC | | |
| (SEQ ID NO: 40) | ATAATATGATCAC | | |
| HPXV 6A-FWD | ATGATACGTACAATG | 163,521 | 175,062 |
| (SEQ ID NO: 41) | ATAATGATACAGTAC | | |
| HPXV 6A-REV | TGATTTTTGCAATTG | | |
| (SEQ ID NO: 42) | TCAGTTAACACAAG | | |
| HPXV 7A-FWD | TACTGTACCCACTAT | 169,035 | 180,578 |
| (SEQ ID NO: 43) | GAATAACGC | | |
| HPXV 7A-REV | GATATCAACATCCAC | | |
| (SEQ ID NO: 44) | TGAAGAAGAC | | |
| HPXV 7B-FWD | ATCTTACCATGTCCT | 175,849 | 187,467 |
| (SEQ ID NO: 45) | CAAATAAATACG | | |
| HPXV 7B-REV | ATAGCTCTAGGTATA | | |
| (SEQ ID NO: 46) | GTCTGCAAG | | |
| HPXV 7C-FWD | GCGAACTCCATTACA | 181,952 | 195,683 |
| (SEQ ID NO: 47) | CAAATATTTG | | |
| HPXV 7D-REV | GATGTTTCTAAATAT | | |
| (SEQ ID NO: 48) | AGGTTCCGTAAGC | | |

The genome sequence of virus isolated from plaques grown from the reactivation assay was confirmed by PCR, restriction digestion, and whole genome sequencing. The PCR analysis was based on the mutated BsaI sites within all but the ITR HPXV fragments. Primer sets were designed to flank each BsaI site in scHPXV YFP-gpt::095 (Table 5). It was confirmed that these primer sets would also amplify a similar region within VACV WR. After PCR amplification of an approximate 1 kb region surrounding these mutated BsaI sites within scHPXV YFP-gpt::095, each reaction was digested with BsaI and the resulting DNA fragments were analyzed by gel electrophoresis. Since no BsaI sites were mutated in VACV (wt), enzymatic digestion successfully digested each PCR product, resulting in a smaller DNA fragment. The PCR products generated from scHPXV YFP-gpt::095 genomic DNA were resistant to BsaI digestion, suggesting that the BsaI recognition site was successfully mutated in these genomes. The primer products for primer set 7C did not result in any amplification of DNA in the scHPXV YFP-gpt::095 PP1 and PP3 samples. To confirm whether this primer set was non-functional or if this area of Fragment 7 did not get assembled into the resulting scHPXV YFP-gpt::095 genome, PCR was performed on the original GA_Frag_7 plasmid DNA and this reaction was also unsuccessful in amplifying a product.

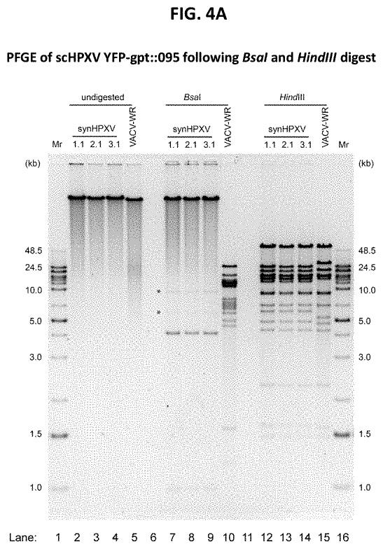

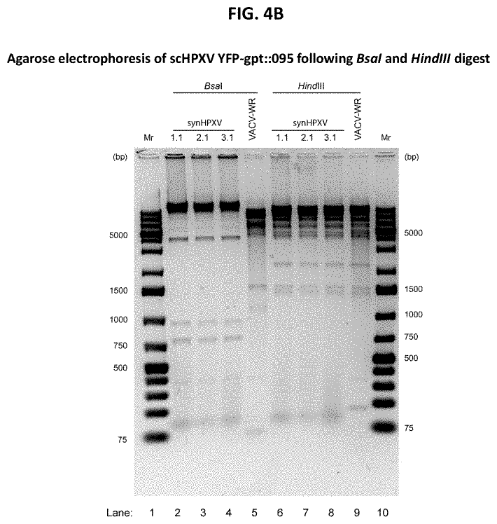

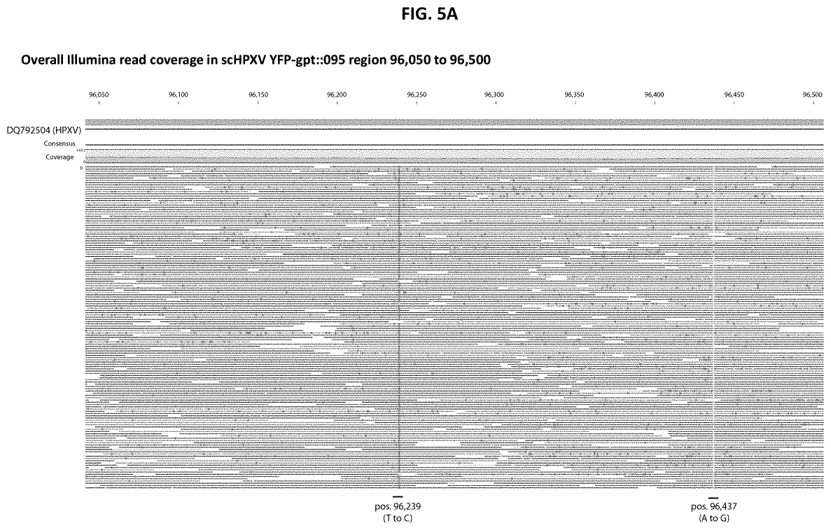

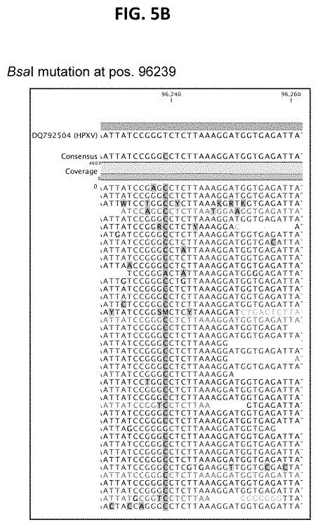

Genomic DNA was next isolated from sucrose-gradient purified scHPXV YFP-gpt::095 genomes, digested with BsaI or HindIII, and separated by agarose gel electrophoresis to confirm that the majority of the BsaI sites in scHPXV YFP-gpt::095 are successfully mutated. Interestingly, undigested genomic DNA from 3 different scHPXV YFP-gpt::095 clones run noticeably slower on a gel compared to VACV, confirming that the genome of scHPXV YFP-gpt::095 (213,305 bp) is larger than VACV-WR (194,711 bp) (FIG. 4A, compare lanes 2-4 with lane 5). The scHPXV YFP-gpt::095 clones were resistant to BsaI digestion, resulting in one large DNA fragment (˜198000 bp) and a smaller DNA fragment at around 4000 bp after separation by PFGE. This is in contrast to the VACV-WR genome, which when digested with BsaI, led to a number of DNA fragments being separated on the gel. Since the expected DNA sizes following digestion of scHPXV YFP-gpt::095 genome with BsaI were relatively small, these digestion products were separated by conventional agarose gel electrophoresis and it was confirmed that the scHPXV YFP-gpt::095 generated the appropriate-sized fragments (FIG. 4B, lanes 2-4). It was also confirmed that scHPXV YFP-gpt::095 produced the correct size of DNA fragments following HindIII digestion, suggesting that these recognitions are maintained during synthesis of the large DNA fragments (FIG. 4A, lanes 12-14; FIG. 4B, lanes 6-8). Overall, in vitro analysis of the scHPXV YFP-gpt::095 genome suggested that reactivation of HPXV from chemically synthesized DNA fragments was successful.

Since HPXV095 encodes the HPXV homolog of the non-essential VACV J2R gene, by co-transfecting Fragment_3 and other HPXV clones into SFV-infected BGMK cells, along with VACV DNA, a variety of hybrid viruses were recovered, validating the selection strategy (FIGS. 10A and 10B). The first hybrid virus (“VACV/HPXV+fragment 3”) was obtained by co-transfecting VACV DNA with HPXV Frag_3 into SFV-infected cells. The green-tagged insertion encodes the YFP-gpt selection marker. Clones 1-3 were obtained by purifying the DNA from this first hybrid genome and transfecting it again, along with HPXV fragments 2, 4, 5, and 7, into SFV-infected cells. PCR primers were designed to target both HPXV and VACV (Table 5) were used to amplify DNA segments spanning the BsaI sites that were mutated in the scHPXV clones. Following PCR amplification, the products were digested with BsaI to differentiate VACV sequences (which cut) from HPXV (which do not cut). The VACV/HPXV hybrids exhibited a mix of BsaI sensitive and resistant sites whereas the reactivated scHPXV YFP-gpt::095 clone was fully BsaI resistant.

Confirmation of scHPXV YFP-Gt:: 095 Genome Sequence by Whole Genome Sequence Analysis

Stocks of HPXV YFP-gpt::095 clones (plaque pick [PP] 1.1, PP 2.1, and PP 3.1]) were prepared and purified over sucrose gradients. Viral DNAs were extracted from each purified virus preparation using proteinase K digestion followed by phenol-chloroform extraction. The amount of dsDNA was determined using a Qubit dsDNA HS assay kit (ThermoFisher Scientific). Each viral genome was sequenced at the Molecular Biology Facility (MBSU) at the University of Alberta. Sequencing libraries were generated using the Nextera Tagmentation system (Epicentre Biotechnologies). Approximately 50 ng of each sample was sheared and library prepped for paired end sequencing (2×300 bp) using an Illumina MiSeq platform with an average read depth of 3,100 reads·nt⁻¹ across the genome and ˜190 reads·nt⁻¹ in the F- and S-hairpins.

### Sequence Assembly, Analysis, and Annotation

Raw sequencing reads were trimmed of low-quality sequence scores and initially mapped to the HPXV reference sequence [GenBank Accession DQ792504] using CLC Genomics Workbench 8.5 software. All nucleotide insertions, deletions, and substitutions within the scHPXV YFP-gpt::095 sequence were verified against the HPXV reference sequence. The Genome Annotation Transfer Utility (GATU) (Tcherepanov V, Ehlers A, Upton C. Genome Annotation Transfer Utility (GATU): rapid annotation of viral genomes using a closely related reference genome. BMC Genomics. 2006; 7:150. Epub 2006/06/15) was used to transfer the reference annotation to the scHPXV genome sequences.

Purified scHPXV YFP-gpt::095 genomes were sequenced using a multiplex approach and an Illumina MiSeq sequencer. The sequence reads were mapped onto the wild-type HPXV (DQ792504) and scHPXV YFP-gpt::095 reference sequences to confirm the presence of specific modifications in the scHPXV YFP-gpt::095 genome. To confirm that the VACV terminal repeat sequences were correctly ligated onto the terminal end of the left ITR, sequencing reads in this area of the genome were analyzed. A string of Cs was added to the beginning of the scHPXV YFP-gpt::095 genome reference sequence to capture all of the sequence reads that mapped in this region. This was done because the program used to assemble the sequence reads will otherwise truncate the display of sequences at the point where the scHPXV YFP-gpt::095 genome reference sequence ends.

It was clear from the mapped reads that although the SapI recognition site was present in the scHPXV YFP-gpt::095 reference genome, all of the sequencing reads lacked this sequence. This confirmed that the approach described herein produces an authentic HPXV sequence at the site where the synthetic hairpin was ligated to the ends of the ITRs. The complete sequence of the VACV WR terminal hairpin loop was also successfully obtained, which proved to be identical to the sequence of the synthetic ssDNA that was ligated onto the TIR ends. Overall, these data suggested that the VACV-WR terminal hairpin loops were successfully ligated onto the HPXV ITR sequences and recovered in the infectious viruses. Moreover, the 1:1 distribution of F- and S-read in each of five viruses suggested that both ends were required to produce a virus.

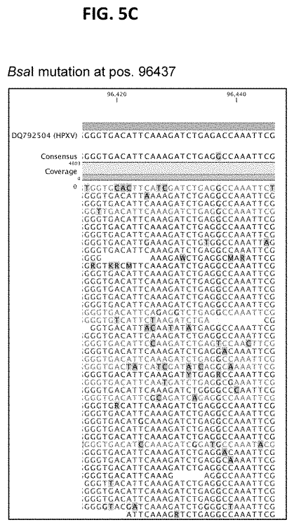

Next, it was verified that each nucleotide substitution to silently mutate the BsaI sites had correctly been incorporated into the scHPXV YFP-gpt::095 genome. Sequencing reads were mapped to the HPXV (DQ792504) reference sequence. The overall Illumina sequencing read coverage in scHPXV YFP-gpt::095 from region 96,050 to 96,500 is shown in FIG. 5A. It was clear from here that there were two conflicts in this region that did not align correctly with reference HIPXV (FIG. 5A, blue and yellow vertical lines). Upon magnification of these regions it was clear that at position 96,239 there was a T to C substitution (FIG. 5B) and at position 96,437 there was an A to G substitution (FIG. 5C) in the scHPXV YFP-gpt::095 genome. It was verified that all of the nucleotide substitutions that were introduced in order to mutate the selected BsaI and AarI recognition sites were created in the scHPXV YFP-gpt::095 genome (Table 2).

Finally, it was determined that the nucleotide substitutions in HPXV044, designed to create unique restriction sites in GA_Frag_2, were also incorporated into the scHPXV YFP-gpt::095 genome. The sequencing reads that map to HPXV044 (region 44,400 to 45,100) showed that within this region there were two regions where the sequencing reads conflicted with that of the sequence in the HPXV YFP-gpt::095 reference sequence. Upon magnification of these regions, it was clear that two T to G substitutions were introduced into the non-coding strand of HPXV044 at positions 44,512 and 45,061, thus creating AvaI and StuI restriction sites in Frag_2. Overall, the sequencing data corroborated the in vitro genomic analysis data and confirmed that scHPXV YFP-gpt::095 was successfully reactivated in SFV-infected cells.

scHPXV YFP-Gpt:: 095 Replicates More Slowly in HeLa Cells Compared to Other Poxviruses

BSC-40, HeLa, and HEL fibroblasts were originally obtained from the American Type Culture Collection. BSC-40 cells are propagated at 37° C. in 5% CO₂ in minimal essential medium (MEM) supplemented with L-glutamine, nonessential amino acids, sodium pyruvate, antibiotics and antimycotics, and 5% fetal calf serum (FCS; ThermoFisher Scientific). HeLa and HEL cells were propagated at 37° C. in 5% CO₂ in Dulbecco's modified Eagle's medium supplemented with L-glutamine, antibiotics and antimycotics, and 10% FCS.

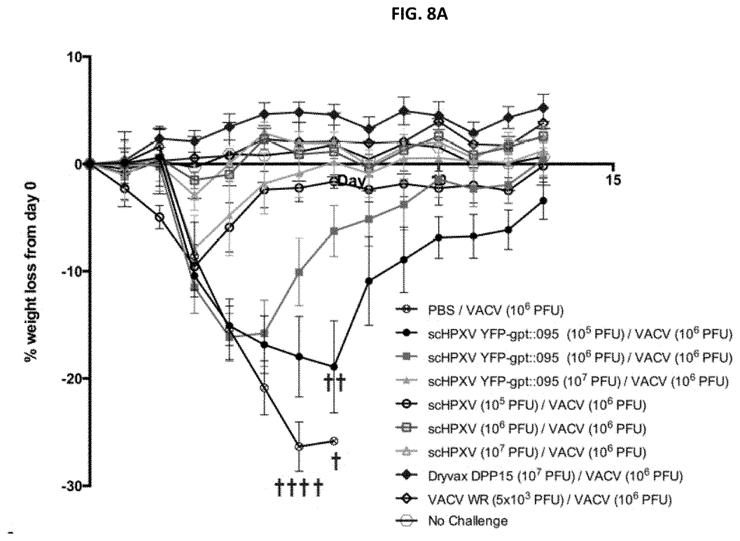

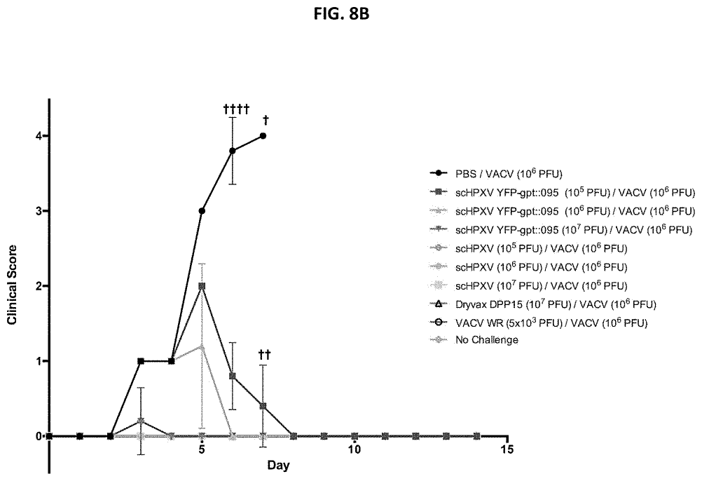

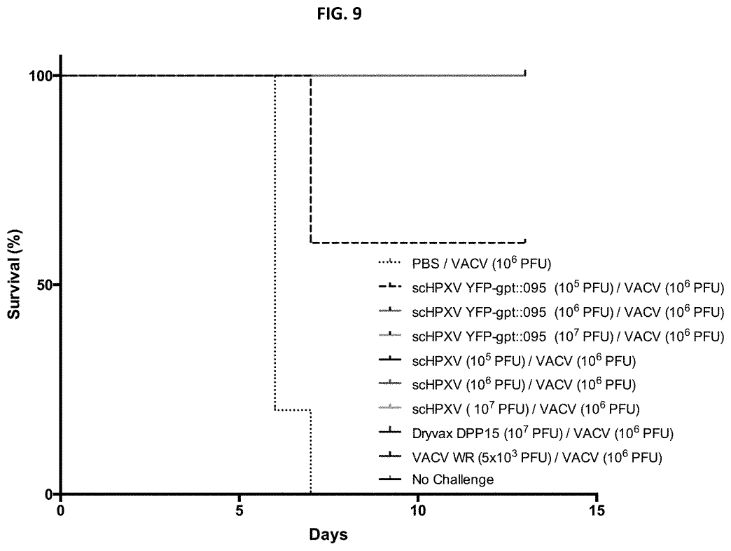

Multi-step growth curves and plaque size measurements were used to evaluate whether scHPXV YFP-gpt::095 replicated and spread in vitro similar to other Orthopoxviruses. Since a natural HPXV isolate was unavailable, the growth of scHPXV YFP-gpt::095 was compared to the prototypic poxvirus, VACV (strain WR), Cowpox virus (CPX), a poxvirus that was closely related to HPXV and a clone of Dryvax virus, DPP15. Monkey kidney epithelial cells (BSC-40), Vero cells, a human carcinoma cell line (HeLa), and primary human fibroblasts cells (HEL) were infected with VACV WR, CPX, DPP15, or scHPXV YFP-gpt::095 at a low MOI and infected cells were harvested over a 72 h time course. In BSC-40 cells, the rate of virus replication and spread was comparable among all viruses tested (FIG. 8A). Importantly, scHPXV YFP-gpt::095 replicated as well as any of the other poxviruses tested. The virus grew to somewhat lower titers on HEL cells and Vero cells, and least well on HeLa cells. In HeLa cells, up to a 1.5-log decrease in virus production was seen compared to other Orthopoxviruses.

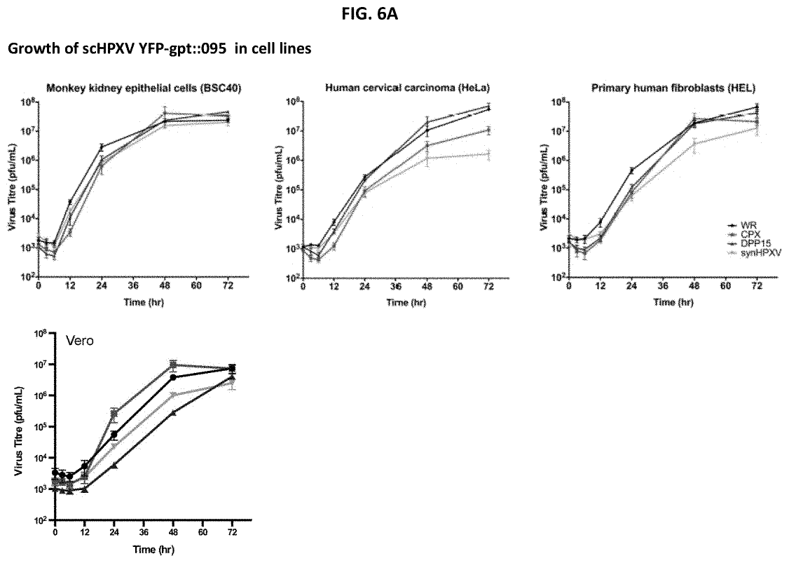

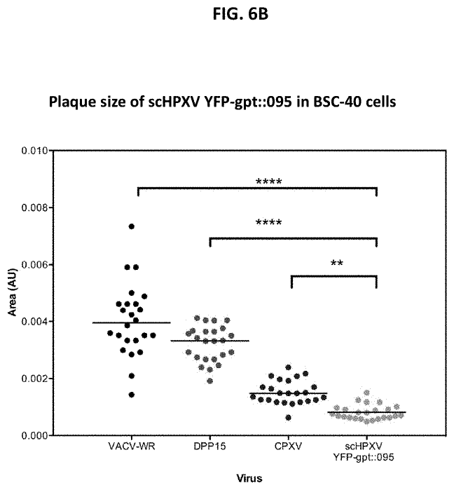

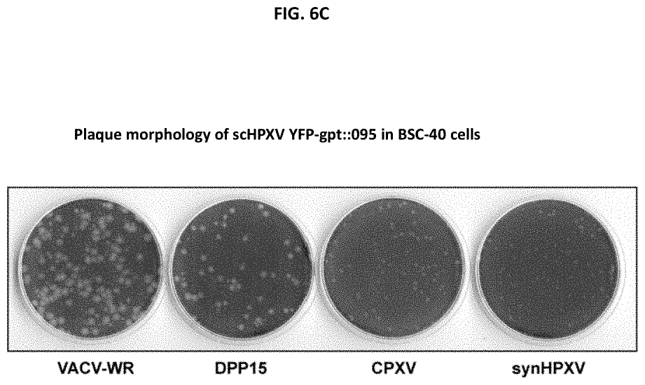

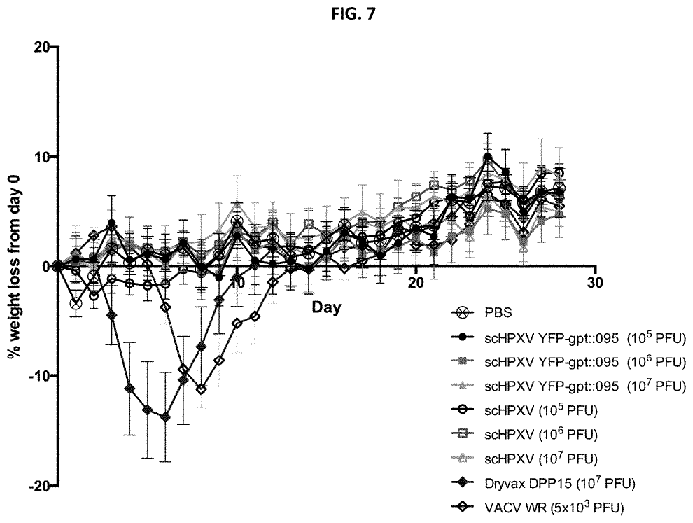

Next, the plaque size of scHPXV YFP-gpt::095 grown in BSC-40 cells was measured. A statistically significant decrease in plaque size of scHPXV YFP-gpt::095 compared to VACV WR and even cowpox virus (FIG. 6B) was observed. Interestingly, in BSC-40 cells, scHPXV YFP-gpt::095 produced the smallest plaques when compared to all other Orthopoxviruses tested (FIG. 6C). Also, while different VACV strains produced extracellular viruses that form smaller secondary plaques, these were not produced by scHPXV YFP-gpt::095 (FIG. 6C). Overall, these data suggested that reactivation of scHPXV YFP-gpt::095 using the system described herein did not introduce any obvious defects in virus replication and spread in vitro when compared to other Orthopoxviruses. Moreover, the plaque size of scHPXV YFP-gpt::095 was similar to that of cowpox virus (CPXV), suggesting that synthetic virus reactivation did not have any deleterious effects on the small plaque phenotype that had previously been observed with other HPXV-like clones (Medaglia M L, Moussatche N, Nitsche A, Dabrowski P W, Li Y, Damon I K, et al. Genomic Analysis, Phenotype, and Virulence of the Historical Brazilian Smallpox Vaccine Strain IOC: Implications for the Origins and Evolutionary Relationships of Vaccinia Virus. Journal of Virology. 2015; 89(23):11909-25).

### Removal of Yfp/Gpt Selection Marker

Following reactivation of the scHPXV YFP-gpt::095, the yfp/gpt selection marker in the HPXV095 locus was removed. To do this, a 1349 bp region of sequence corresponding to nucleotide positions 91573 to 92921 in HPXV (DQ792504) was synthesized (ThermoFisher Scientific) (SEQ ID NO: 51). This fragment included approximately 400 bp of homology flanking either side of the wt HPXVO95/J2R gene. This sequence of DNA was cloned into a commercial vector provided by GeneArt. To replace the yfp/gpt cassette with the HPXV095 gene sequence, BSC-40 cells were infected with scHPXV YFP-gpt::095 at a MOI of 0.5 and then transfected, 2 h later, with 2 μg of linearized plasmid containing the wtHPXV095 sequence using Lipofectamine 2000 (ThermoFisher Scientific). The virus recombinants were harvested 48 h post infection and recombinant viruses (scHPXV (wt)) were isolated using three rounds of non-fluorescent plaque purification under agar. PCR was used to confirm the identity of the scHPXV (wt) using primers that flanked the HPXV095 gene locus. The primers used to confirm the correct replacement of the HPXV095 gene are HPXV095_check-FWD 5′-CCTATTAGATACATAGATCCTCGTCG-3′ (SEQ ID NO: 52) and HPXV095_check-REV 5′-CGGTTTATCTAACGACACAACATC-3′ (SEQ ID NO: 53).

Growth Properties of scHPXV (Wt) Versus scHPXV YFP-Gpt::095

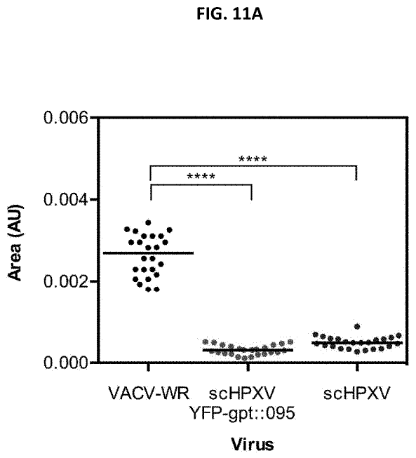

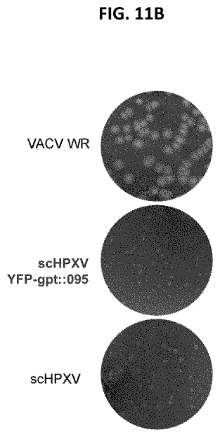

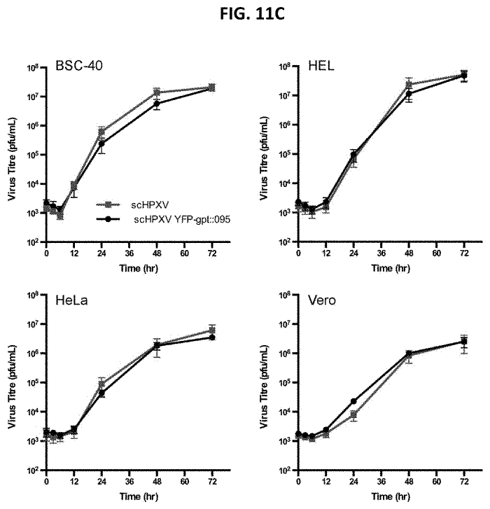

In experiments performed as described above, scHPXV(wt) shows growth properties not significantly different from scHPXV YFP-gpt::095 in vitro (FIG. 11A-C). A statistically significant decrease in plaque size of scHPXV(wt) compared to VACV WR was observed (FIG. 11A). scHPXV (wt), like scHPXV YFP-gpt::095, did not produce extracellular viruses (FIG. 11B) and there were no significant differences in the growth of scHPXV (wt) and scHPXV YFP-gpt::095 on BSC-40 cells, HEL cells, HeLA cells, and Vero cells (FIG. 11C). The finding that scHPXV(wt) did not produce extracellular viruses was of relevance given that this property affects virulence.

Determination of the Virulence of scHPXV (Wt) in a Murine Intranasal Model

The toxicity effects of scHPXV (wt) were determined in this study. For this experiment, 6 groups of Balb/c mice were administered 3 different doses of scHPXV (ΔHPXV_095/J2R) or scHPXV (wt) described in the Examples and compared to a PBS control group as well as a VACV (WR) control group and a VACV (Dryvax strain DPP15) control group (9 treatment groups in total). There were 3 additional mice included in this experiment that did not receive any treatment for the duration of the study. All mice were sampled for blood at predetermined points throughout the experiment and the additional mice served as a baseline for serum analysis.

Prior to inoculation of Balb/c mice, all virus strains were grown in BSC-40 cells (African green monkey kidney), harvested by trypsinization, washed in PBS, extracted from cells by dounce homogenization, purified through a 36% sucrose cushion by ultracentrifugation, resuspended in PBS, and titered such that the final concentrations were: 1) VACV (WR)—5×10⁵ PFU/ml; 2) VACV (DPP15)—10⁹ PFU/ml; 3) scHPXV (ΔHPXV_095/J2R)—10⁷ PFU/ml, 10⁸ PFU/ml, and 10⁹ PFU/ml and 4) scHPXV (wt)—10⁷ PFU/ml, 10⁸ PFU/ml, and 10⁹ PFU/ml.

The scHPXV doses chosen for this study (10⁵ PFU/dose, 10⁶ PFU/dose, and 10⁷ PFU/dose) were based on previous studies using known vaccine strains of VACV, including Dryvax and IOC (Medaglia M L, Moussatche N, Nitsche A, Dabrowski P W, Li Y, Damon I K, et al. Genomic Analysis, Phenotype, and Virulence of the Historical Brazilian Smallpox Vaccine Strain IOC: Implications for the Origins and Evolutionary Relationships of Vaccinia Virus. Journal of Virology. 2015; 89(23):11909-25; Qin L, Favis N, Famulski J, Evans D H. Evolution of and evolutionary relationships between extant vaccinia virus strains. Journal of Virology. 2015; 89(3):1809-24).

Since weight loss was used as a measurement of virulence in mice, VACV (strain WR) was administered intranasally at a dose of 5×10³ PFU, which led to approximately 20-30% weight loss. The VACV Dryvax clone, DPP15, was also administered intranasally at 10⁷ PFU/dose, so that the virulence of this well-known Smallpox vaccine could be directly compared to scHPXV (wt). Mice were purchased from Charles River Laboratories and once received, were acclimatized to their environment for at least one week prior to virus administration.

Each mouse received a single dose of virus (˜10 ul) administered via the intranasal injection while under anesthesia. Mice were monitored for signs of infection, such as swelling, discharge, or other abnormalities every day for a period of 30 days. Each mouse was specifically monitored for weight loss every day after virus administration. Mice that lose more than 25% of their body weight in addition to other morbidity factors were subjected to euthanasia in accordance with our animal health care facility protocols at the University of Alberta.

Even at the highest doses of scHPXV tested, there may be no overt signs of illness in Balb/c mice. The VACV strains most closely related to scHPXV, old South American viruses, in some cases produced no disease at 10⁷ PFU (Medaglia M L, Moussatche N, Nitsche A, Dabrowski P W, Li Y, Damon I K, et al. Genomic Analysis, Phenotype, and Virulence of the Historical Brazilian Smallpox Vaccine Strain IOC: Implications for the Origins and Evolutionary Relationships of Vaccinia Virus. Journal of Virology. 2015; 89(23):11909-25). It was impractical to test much higher doses than this due to the difficulty of making purified stocks with titers in excess of 10⁹ PFU/mL.

Determination of Whether scHPXV Confers Immune Protection Against a Lethal VACV-WR Challenge

Mice that appeared to have been unaffected by the initial virus administration described in the Example continued to gain weight normally throughout the experiment. Thirty days post virus inoculation, mice were subsequently challenged with a lethal dose of VACV-WR (10⁶ PFU/dose) via intranasal inoculation. Mice were closely monitored for signs of infection as described above. Mice were weighed daily and mice that lost greater than 25% of their body weight in addition to other morbidity factors were subjected to euthanasia. We expected that mice inoculated with PBS prior to administration of a lethal dose of VACV-WR showed signs of significant weight loss and other morbidity factors within 7-10 days post inoculation. Approximately 14 days post lethal challenge with VACV-WR, all mice were euthanized and blood was collected to confirm the presence of VACV-specific neutralizing antibodies in the serum by standard plaque reduction assays.

### Example 2. Synthetic Chimeric VACV (scVACV)

Synthetic Chimeric VACV ACAM2000 Containing VACV WR Strain Hairpin and Duplex Sequence (scVACVACAM2000-WR DUP/HP)

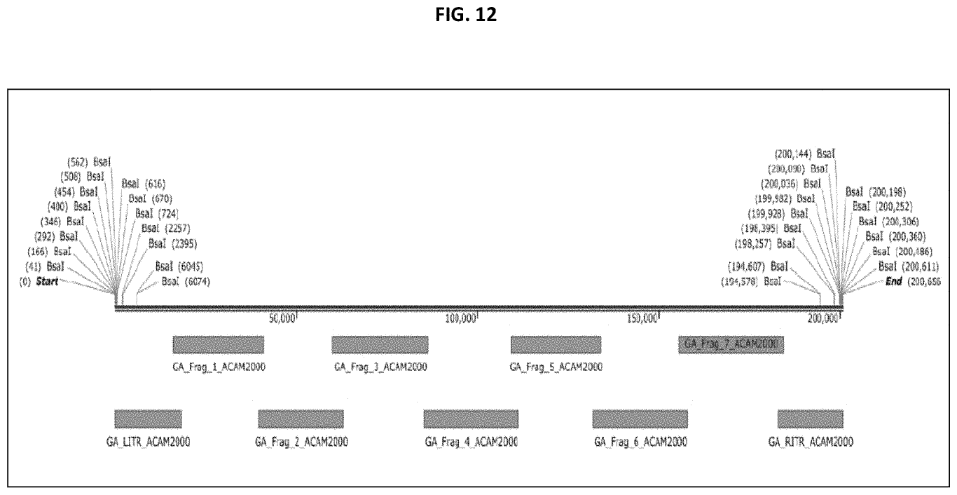

The design of the scVACV genome was based on the previously described genome sequence for VACV ACAM2000 [GenBank accession AY313847] (Osborne J D et al. Vaccine. 2007; 25(52):8807-32). The genome was divided into 9 overlapping fragments (FIG. 12). These fragments were designed so that they shared at least 1.0 kbp of overlapping sequence (i.e. homology) with each adjacent fragment, to provide sites where homologous recombination will drive the assembly of full-length genomes (Table 4). These overlapping sequences provided sufficient homology to accurately carry out recombination between the co-transfected fragments (Yao X D, Evans D H. Journal of Virology. 2003; 77(13):7281-90).

TABLE 4
The VACV ACAM2000 genome fragments used in this study.
The size and the sequence within the VACV ACAM2000
genome [GenBank Accession AY313847] are described.

| Fragment Name | Size (bp) | Sequence |
|---|---|---|
| GA_LITR | 18,525 | SEQ ID NO: 54 |
| ACAM2000 | | |
| GA_FRAG_1 | 24,931 | SEQ ID NO: 55 |
| ACAM2000 | | |
| GA_FRAG_2 | 23,333 | SEQ ID NO: 56 |
| ACAM2000 | | |
| GA_FRAG_3 | 26,445 | SEQ ID NO: 57 |
| ACAM2000 | | |
| GA_FRAG_4 | 26,077 | SEQ ID NO: 58 |
| ACAM2000 | | |
| GA_FRAG_5 | 24,671 | SEQ ID NO: 59 |
| ACAM2000 | | |
| GA_FRAG_6 | 25,970 | SEQ ID NO: 60 |
| ACAM2000 | | |
| GA_FRAG_7 | 28,837 | SEQ ID NO: 61 |
| ACAM2000 | | |
| GA_RITR | 17,640 | SEQ ID NO: 62 |
| ACAM2000 | | |

To assist with sub-cloning of these fragments, AarI and BsaI restriction sites were silently mutated in all the fragments, except for the two ITR-encoding fragments. The BsaI restriction sites in the two ITR-encoding fragments were not mutated, in case these regions contain nucleotide sequence-specific recognition sites that are important for efficient DNA replication and concatamer resolution.

A YFP/gpt cassette under the control of a poxvirus early late promoter was introduced into the thymidine kinase locus, so that reactivation of VACV ACAM2000 (VACV ACAM2000 YFP-gpt::105) was easy to visualize under a fluorescence microscope. The gpt locus also provided a potential tool for selecting reactivated viruses using drug selection.

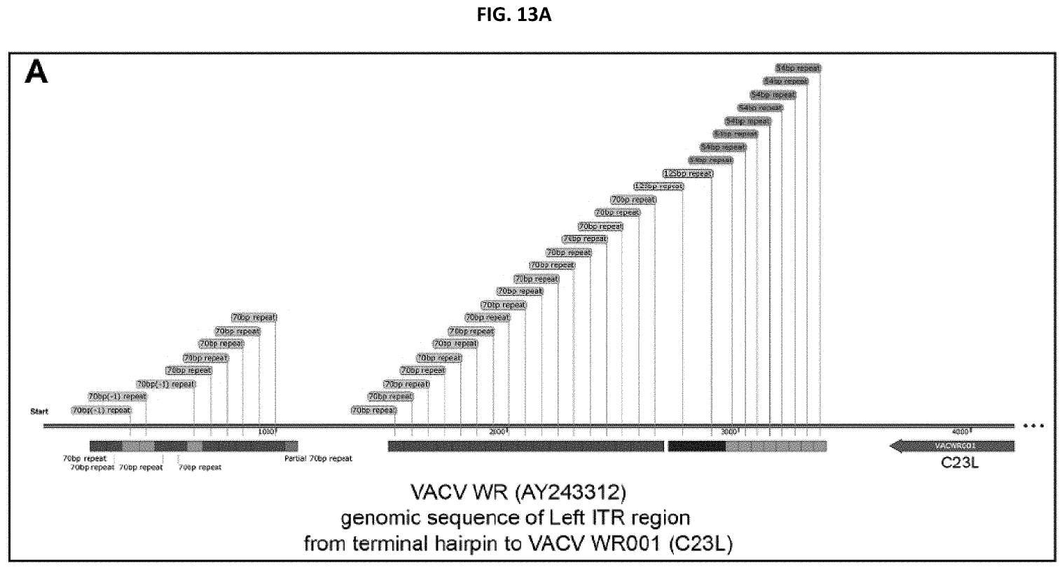

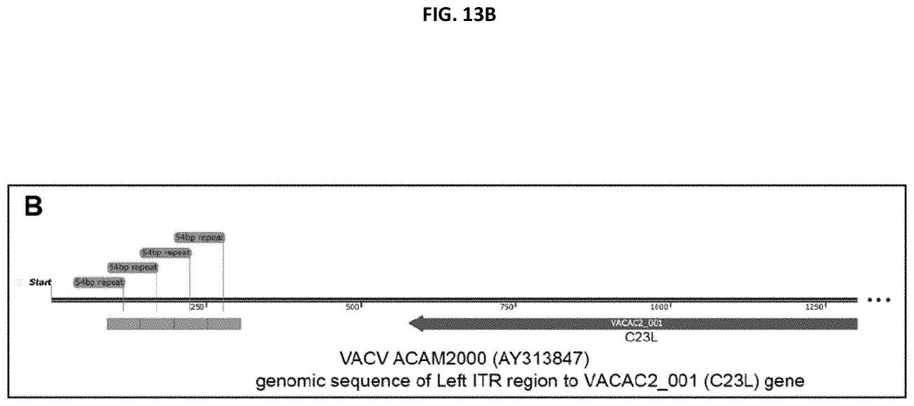

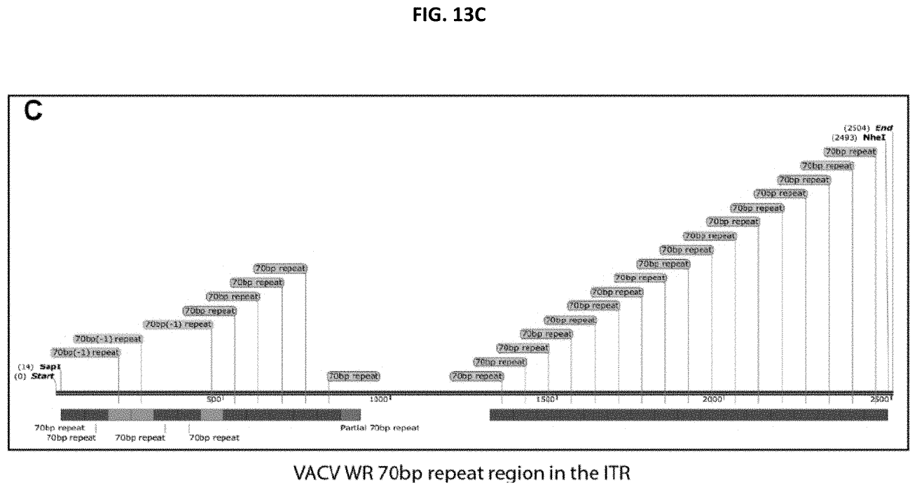

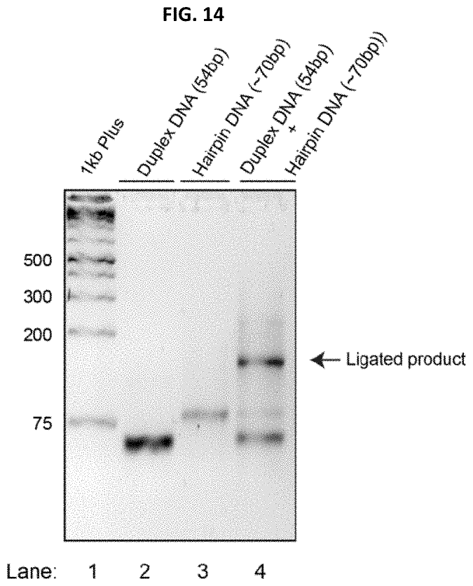

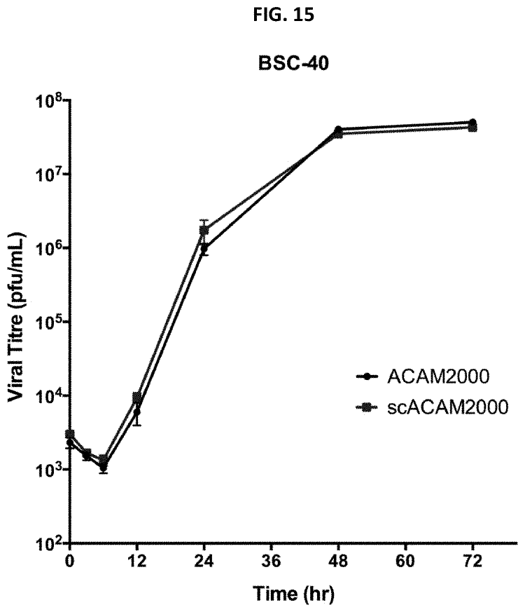

Traditionally, the terminal hairpins have been difficult to clone and sequence, hence, it is not surprising that the published sequence of the VACV ACAM2000 genome is not complete. Upon inspection of the very terminal region of the published VACV ACAM2000 strain, there appeared to be some differences between ACAM2000 and the very well characterized VACV WR strain (Genbank Accession #AY243312) (FIG. 12). In the WR strain, there are 70 bp tandem repeat sequences immediately downstream of the covalently closed hairpin loop that is located at the terminal 5′ and 3′ termini of the VACV genome. These are followed by two 125 bp repeat sequences and eight 54 bp repeat sequences (FIG. 13A). In the published VACV ACAM2000 sequence, however, only four 54 bp repeat sequences were identified. The presence of the 70 bp, 125 bp, and 54 bp repeat sequences was confirmed in a wild-type isolate of VACV ACAM2000 after sequencing (using Illumina), indicating that the current published sequence of ACAM2000 is incomplete. Due to the short read lengths of the Illumina reads (<300 nucleotides), the inventors were unable to accurately determine what the actual ACAM2000 genomic sequence was in this 3 kbp. Instead, the inventors decided to recreate a VACV ACAM2000 virus that had a similar sequence to VACV WR from the terminal hairpin to just before the stop codon of the C23L gene (FIG. 12). This included both the 125 bp and 54 bp tandem repeat sequences that, although not included in the published ACAM2000 sequence, were detected when next generation Illumina sequencing of the wtVACV ACAM2000 was performed. At the 5′ termini of the modified VACV ACAM2000 left and right ITR fragments an NheI restriction site was also included, that would allow to directly attach the 70 bp tandem repeat sequence to the ITR ends (discussed above). The F and S terminal hairpin loop sequences of the wtVACV WR strain are shown in SEQ ID NO: 11 and 12, respectively.

Synthetic Chimeric VACV ACAM2000 Containing VACV ACAM2000 Strain Hairpin and Duplex Sequence (scVACVACAM2000-ACAM2000 DUP/HP)

The design of the scVACV genome was based on the previously described genome sequence for VACV ACAM2000 [GenBank accession AY313847] (Osborne J D et al. Vaccine. 2007; 25(52):8807-32). The genome was divided into 9 overlapping fragments (FIG. 12). These fragments were designed so that they shared at least 1.0 kbp of overlapping sequence (i.e. homology) with each adjacent fragment, to provide sites where homologous recombination will drive the assembly of full-length genomes (Table 4). These overlapping sequences provided sufficient homology to accurately carry out recombination between the co-transfected fragments (Yao X D, Evans D H. Journal of Virology. 2003; 77(13):7281-90).

To assist with sub-cloning of these fragments, AarI and BsaI restriction sites were silently mutated in all the fragments, except for the two ITR-encoding fragments. The BsaI restriction sites in the two ITR-encoding fragments were not mutated, in case these regions contain nucleotide sequence-specific recognition sites that are important for efficient DNA replication and concatemer resolution.

A YFP/gpt cassette under the control of a poxvirus early late promoter was introduced into the thymidine kinase locus, so that reactivation of VACV ACAM2000 (VACV ACAM2000 YFP-gpt::105) was easy to visualize under a fluorescence microscope. The gpt locus also provided a potential tool for selecting reactivated viruses using drug selection.

The F and S terminal hairpin loop sequences of the wtVACV ACAM2000 are shown in SEQ ID NO: 118 and 117, respectively.

Ligation of the VACV WR F and S Terminal Hairpin Loops onto the VACV ACAM2000 Right and Left ITR Fragments

A 70 bp repeat fragment that was identical to the VACV WR strain was synthesized (FIG. 13A; SEQ ID NO: 63). SapI and NheI restriction sites were included at the 5′ and 3′ terminus of the 70 bp tandem repeat fragment to facilitate the ligation onto the VACV WR hairpin sequence and the VACV ACAM2000 right and left ITR fragments, respectively. Before the VACV WR terminal hairpin loops could be ligated onto the 70 bp tandem repeat fragment, the loop had to be extended an additional 58 bp using a duplex sequence synthesized by IDT. This was due to the extra sequence being immediately downstream of the concatemer resolution site, prior to the first 70 bp repeat sequence found in VACV strain WR. The duplex sequence was produced by synthesizing two single-stranded DNA molecules that, when annealed together, would produce a duplex DNA molecule with a 5′-TGT overhang at the 5′ end and a 5′-GGT overhang at the 3′ end (SEQ ID NO: 64 and SEQ ID NO: 65). Since the VACV WR F and S terminal hairpin loops generate a 3′-ACA overhang at their terminal loops, the 58 bp duplex was ligated to the hairpins to generate an ˜130 bp terminal hairpin loop that looked identical to the sequence found in the VACV WR strain up until the beginning of the 70 bp repeat sequence. This hairpin/duplex fragment was gel purified and then subsequently ligated onto the SapI digested end of the 70 bp repeat fragment. Digesting the 70 bp tandem repeat fragment with SapI created a three base overhang (5′-CCA), complementary to the 5′GGT overhang in the terminal hairpin/duplex structure. The 70 bp tandem repeat was mixed with either an F terminal hairpin/duplex structure or a S terminal hairpin/duplex structure at a ˜5-fold molar excess relative to the 70 bp tandem repeat fragment in the presence of DNA ligase. This produced an upward shift in the DNA electrophoresis gel compared to the 70 bp only reaction, indicating that the terminal hairpin/duplex was successfully ligated onto the 70 bp tandem repeat fragment.

This terminal hairpin/duplex/70 bp tandem repeat fragment was subsequently ligated onto the 70 bp ACAM2000 left or right ITR fragment that had been previously modified at their terminal ends to include the NheI restriction site. When this fragment was digested, a 5′-CTAG overhang was left at their 5′ termini. At the 3′ terminus of the 70 bp tandem repeat fragment, the NheI site is used to directly ligate this fragment to the LITR and RITR regions of the VACV ACAM2000 DNA fragments. Following digestion of the VACV ACAM2000 left and right ITR fragments, the S terminal hairpin/duplex/70 bp tandem repeat fragment or the F terminal hairpin/duplex/70 bp tandem repeat fragment were separately ligated to either the left or right ITR fragment using DNA ligase at a 1:1 molar ratio overnight at 16° C. The DNA ligase was subsequently heat inactivated at 65° C. prior to being transfected into Shope Fibroma virus (SFV)-infected BGMK cells.

Ligation of the VACV ACAM2000 F and S Terminal Hairpin Loops onto the VACV ACAM2000 Right and Left ITR Fragments

A 70 bp repeat fragment that was identical to the VACV ACAM2000 strain was synthesized. SapI and NheI restriction sites were included at the 5′ and 3′ terminus of the 70 bp tandem repeat fragment to facilitate the ligation onto the VACV ACAM2000 hairpin sequence and the VACV ACAM2000 right and left ITR fragments, respectively. Before the VACV ACAM2000 terminal hairpin loops could be ligated onto the 70 bp tandem repeat fragment, the loop had to be extended an additional 58 bp using a duplex sequence synthesized by IDT Technologies. This was due to the extra sequence being immediately downstream of the concatemer resolution site, prior to the first 70 bp repeat sequence found in VACV strain ACAM2000. The duplex sequence was produced by synthesizing two single-stranded DNA molecules that, when annealed together, would produce a duplex DNA molecule with a 5′-TGT overhang at the 5′ end and a 5′-GGT overhang at the 3′ end (SEQ ID NO: 119 and SEQ ID NO: 120). Since the VACV ACAM2000 F and S terminal hairpin loops generate a 3′-ACA overhang at their terminal loops, the 58 bp duplex was ligated to the hairpins to generate an ˜130 bp terminal hairpin loop. This hairpin/duplex fragment was gel purified and then subsequently ligated onto the SapI digested end of the 70 bp repeat fragment. Digesting the 70 bp tandem repeat fragment with SapI created a three-base overhang (5′-CCA), complementary to the 5′GGT overhang in the terminal hairpin/duplex structure. The 70 bp tandem repeat was mixed with either an F terminal hairpin/duplex structure or a S terminal hairpin/duplex structure at a ˜5-fold molar excess relative to the 70 bp tandem repeat fragment in the presence of DNA ligase. This produced an upward shift in the DNA electrophoresis gel compared to the 70 bp only reaction, indicating that the terminal hairpin/duplex was successfully ligated onto the 70 bp tandem repeat fragment.

This terminal hairpin/duplex/70 bp tandem repeat fragment was subsequently ligated onto the ACAM2000 left or right ITR fragment that had been previously modified at their terminal ends to include the NheI restriction site. When this left or right ITR fragment was digested, a 5′-CTAG overhang was left at their 5′ termini. At the 3′ terminus of the 70 bp tandem repeat fragment, the NheI site is used to directly ligate this fragment to the LITR and RITR regions of the VACV ACAM2000 DNA fragments. Following digestion of the VACV ACAM2000 left and right ITR fragments, the S terminal hairpin/duplex/70 bp tandem repeat fragment or the F terminal hairpin/duplex/70 bp tandem repeat fragment were separately ligated to either the left or right ITR fragment using DNA ligase at a 1:1 molar ratio overnight at 16° C. The DNA ligase was subsequently heat inactivated at 65° C. prior to being transfected into Shope Fibroma virus (SFV)-infected BGMK cells.

### Preparation of the VACVACAM2000 Overlapping DNA Fragments

Each of the VACV ACAM2000 overlapping DNA fragments in Table 4 were cloned into a plasmid provided from GeneArt using the restriction enzyme I-SceI. Prior to transfection of these synthetic DNA fragments into BGMK cells, the plasmids were digested with I-SceI and the products were run on a gel to confirm that the DNA fragments were successfully linearized. Following digestion at 37° C. for 2 h, the reactions were subsequently heat-inactivated at 65° C. Samples were stored on ice or at 4° C. until the terminal hairpin/duplex/70 bp tandem repeat/ITR fragments were created (as described above).

Reactivation of scVACV ACAM2000-WR DUP/HP or scVACV ACAM2000-ACAM2000 DUP/HP in Shope Fibroma Virus-Infected Cells

SFV strain Kasza and BSC-40 were originally obtained from the American Type Culture Collection. Buffalo green monkey kidney (BGMK) cells were obtained from G. McFadden (University of Florida). BSC-40 and BGMK cells are propagated at 37° C. in 5% CO₂ in minimal essential medium (MEM) supplemented with L-glutamine, nonessential amino acids, sodium pyruvate, antibiotics and antimycotics, and 5% fetal calf serum (FCS; ThermoFisher Scientific).

Buffalo green monkey kidney (BGMK) cells were grown in MEM containing 60 mm tissue-culture dishes until they reached approximately 80% confluency. Cells were infected with Shope Fibroma Virus (SFV) in serum-free MEM at a MOI of 0.5 for 1 h at 37° C. The inoculum was replaced with 3 ml of warmed MEM containing 5% FCS and returned to the incubator for an additional hour. Meanwhile, transfection reactions were set up as follows. After approximately 2 h at 37° C., the linearized VACV ACAM2000 fragments were transfected (using Lipofectamine 2000) into the SFV-infected BGMK cells at molar equivalents based on the length of each fragment that comprised the VACV ACAM2000 genome. Different amounts of total DNA were tried and 5, 6, and 7.5 μg of DNA were able to successfully reactivate ACAM2000 from these overlapping DNA fragments. The complexes were incubated at room temperature for 10 minutes and then added dropwise to the BGMK cells previously infected with SFV. Approximately 24 h post infection, the media was replaced with fresh MEM containing 5% FCS. The cells were cultured for an additional 4 days (total of 5 days) at 37° C.

Virus particles were recovered by scraping the infected cells into the cell culture medium and performing three cycles of freezing and thawing. The crude extract was diluted 10² in serum-free MEM and 4 ml of the inoculum is plated on 9-16 150 mm tissue culture plates of BSC-40 cells to recover reactivated scVACV ACAM2000 YFP-gpt::105. One hour post infection, the inoculum was replaced with MEM containing 5% FCS and 0.9% Noble Agar. Yellow fluorescent plaques were visualized under an inverted microscope and individual plaques were picked for further analysis. scVACV ACAM2000 YFP-gpt::105 plaques were plaque purified three times with yellow fluorescence selection.

After 4 days, the infected plates containing both SFV and VACV ACAM2000 clones were harvested, followed by three freeze thaw cycles to release virus, and then serially diluted and plated onto BSC-40 cells, which preferentially promote growth of the VACV ACAM2000 viruses compared to the SFV viruses. Three rounds of plaque purification were performed followed by a bulkup of the virus stocks in 10-150 mm tissue culture plates. The virus was subsequently lysed from these cells and separated on a 36% sucrose cushion, followed by further purification on a 24%-40% sucrose density gradient. Genomic DNA was isolated from these purified genomes and next generation Illumina sequencing was performed to confirm the sequence of the synthetic virus genomes.

### Growth Properties Compared to Wild Type ACAM2000 Virus

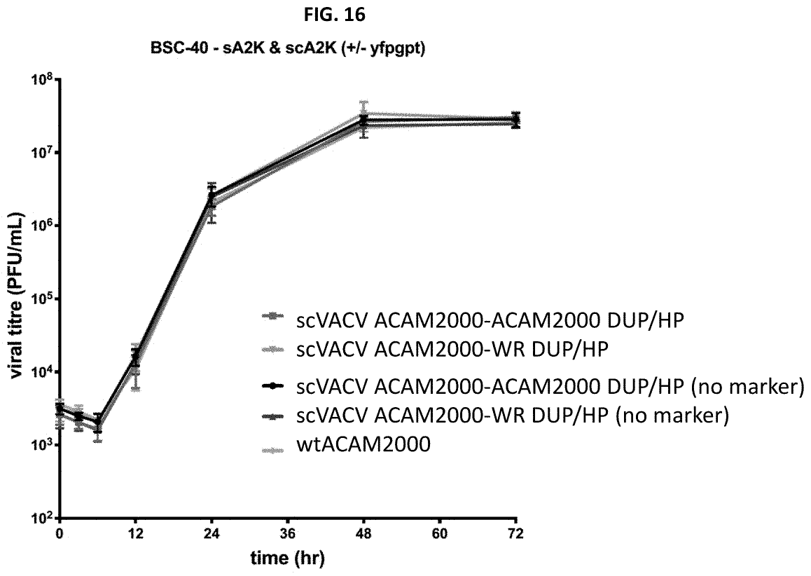

In vitro multi-step growth curves of the isolated synthetic chimeric VACV ACAM2000-WR DUP/HP, scVACV ACAM2000-ACAM2000 DUP/HP and the wild type VACV ACAM2000 virus were performed in monkey kidney epithelial (BSC-40) cells. The cells were infected at a multiplicity of infection 0.03, the virus was harvested at the indicated times (3 h, 6 h, 12 h, 21 h, 48 h and 72 h), and the virus was titrated on BSC-40 cells. The data shown on FIG. 16 represent three independent experiments. As shown on FIG. 16, scVACV ACAM2000 and wtVACV ACAM2000-WR DUP/HP viruses grew with indistinguishable growth kinetics over a 72 h period.

A comparison between the growth curves of scVACV ACAM2000-WR DUP/HP (YFP-gpt marker), scVACV ACAM2000-ACAM2000 DUP/HP (YFP-gpt marker), scVACV ACAM2000-WR DUP/HP (no marker) (YFP-gpt marker replaced with J2R gene sequence), scVACV ACAM2000-ACAM2000 DUP/HP (no marker) (YFP-gpt marker replaced with J2R gene sequence) and wtVACV ACAM2000, shows that there is statistically no difference in the growth properties of these viruses as compared to the wtACAM2000 VACV (FIG. 16).

Confirmation of scVACV ACAM2000-WR DUP/HP YFP-Gpt::105 Genome Sequence by PCR and Restriction Fragment Analysis

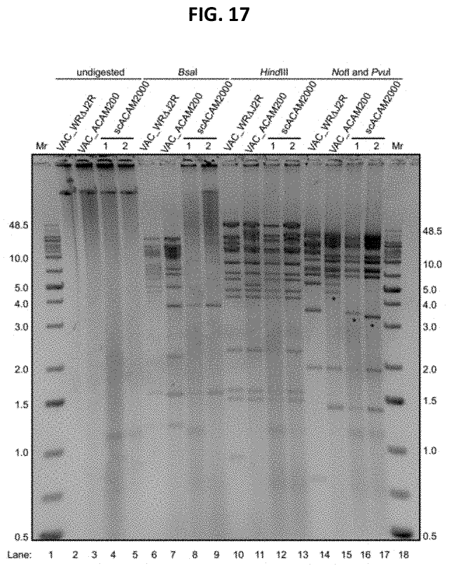

Further analysis of scVACV ACAM2000 YFP-gpt::105 genomes by restriction digestion followed by pulse-field gel electrophoresis (PFGE) was carried out on genomic DNA isolated using sucrose gradient purification (Yao X D, Evans D H. Methods Mol Biol. 2004; 269:51-64). Two independent scVACV ACAM2000-WR DUP/HP clones plus a VACV_WRAJ2R control where the J2R gene sequence has been replaced with a YFP-gpt marker, and a wtVACV ACAM2000 control (VAC_ACAM2000) were purified and then left either undigested, digested with BsaI, HindIII, or Not and PvuI. The isolated genomic DNA from both scVACV ACAM2000-WR DUP/HP and wtVACV ACAM2000 were digested with BsaI and HindIII. Since most of the BsaI sites in the scVACV ACAM2000 genome had been silently mutated, a mostly intact ˜200 kbp fragment was observed following BsaI digestion (FIG. 17, lanes 8 and 9). This is unlike the wtVACV ACAM2000 and wtVACV WR control (VAC_WRAJ2R) genomes, which had been extensively digested when treated with BsaI (FIG. 17, lanes 6 and 7). To confirm that the scVACV ACAM2000-WR DUP/HP genome could still be digested with another enzyme, these genomes were digested with HindIII, which produced numerous bands from the scVACV ACAM2000-WR DUP/HP clones (FIG. 17, lanes 12 and 13). To confirm the presence of the 70 bp tandem repeat elements within the ITR regions, the genomic DNA was digested with Not and PvuI (FIG. 17, lanes 14 to 17).

In the wtVACV WR control (VAC_WRAJ2R) sample, a band at about 3.6 kbp (marked with asterisks) was detected, which encompasses all of the 70 bp tandem repeats in the WR strain of VAC. Given that the VACV WR strain used as a template to design the synthesis of the ITR repeat elements, it was expected that some bands were detected in the NotI/PvuI treated scVACV ACAM2000-WR DUP/HP clones at close to the same size as what was seen in strain WR. When the two scVACV ACAM2000-WR DUP/HP clones were compared differences in the size of this region were observed, suggesting that not all of the 70 bp repeats were incorporated into each reconstructed genome (FIG. 17, lanes 16 and 17). This is not unexpected, given that others have shown that these repeat elements can expand and contract under selective pressure in cell culture (Paez and Esteban 1988).

Overall, in vitro analysis of the scVACV-WR DUP/HP ACAM2000 YFP-gpt::105 genome suggested that reactivation of VACV-WR DUP/HP ACAM2000 from chemically synthesized DNA fragments was successful and that scVACV-WR DUP/HP ACAM2000 virus behaved in vitro like the wtVACV ACAM2000 virus.

Confirmation of scVACV ACAM2000 YFP-Gpt::105 Genome Sequence by Whole Genome Sequence Analysis

Two clones of scVACV ACAM2000-WR DUP/HP and two clones of scVACV ACAM2000-ACAM2000 DUP/HP were sequenced. The Illumina reads were de novo assembled using CLC Genomics Workstation (version 11) with a word size of 35 or 61. Assembled contigs were then imported into Snapgene software and aligned onto a reference sequence of the expected scACAM2000 sequence based on the synthetic fragments that were provided by GeneArt.

For clone 1 of scVACV ACAM2000-WR DUP/HP, contig 1 was 16,317 bp, and corresponded to most of the ITR region (except for the tandem repeat sequences. Contig 2 was 167,020 bp, and aligned with the central conserved region of the genome (nucleotide positions 19,467 to 186,486). For clone 2 of scVACV ACAM2000-WR DUP/HP, contig 3 was 16,322 bp, and corresponded to most of the ITR region (except for the tandem repeat sequences. Contig 1 was 167,020 bp, and aligned with the central conserved region of the genome (nucleotide positions 19,467 to 186,486). There was a single nucleotide substitution (C to A) at nucleotide position 136791 of the contig of clone 2. This corresponded to nucleotide position 156,256 in the scACAM2000 genome sequence and resulted in an amino acid change from an Asp to Tyr in VAC_ACAM2000_177 (A41L).

For clone 1 of scVACV ACAM2000-ACAM2000 DUP/HP, contig 1 was 167,020 bp, and aligned with the central conserved region of the genome (nucleotide positions 19,469 to 186,488). Contig 2 was 16,150 bp, and corresponded to most of the ITR region (except for tandem repeat sequences). When this contig was mapped to the reference genome in Snapgene, gaps in the sequence were observed at positions 2633 to 3417 and nucleotide positions 15,175 to 15220. The first gap region corresponds to the 54 bp repeat region and it is most likely due to the inability to accurately assemble these regions using de novo assembly tools. Mapping of the raw Illumina reads directly to the reference genome did not result in any gaps within either region. For clone 2 of scVACV ACAM2000-ACAM2000DUP/HP, contig 1 was 16,075 bp, and corresponded to most of the ITR region (except the tandem repeat sequences). Contig 2 was 167,078 bp and aligned with the central conserved region of the genome (nucleotide positions 19,469 to 186,546). There was a gap observed in contig 2 from nucleotide position 15,176 to 15,220. Mapping of the raw Illumina reads directly to the reference genome did not result in any gaps within this region, however. Neither sequenced clone of scVACV ACAM2000-ACAM2000 DUP/HP displayed any other nucleotide mutations at any position within the genome.

The Illumina reads were also mapped to a reference map in CLC Genomics. The Illumina reads covered the full length of the reference sequence with an average coverage of 1925 and 2533, for clone 1 and 2 of scVACV ACAM2000-WR DUP/HP, respectively, and an average coverage of 2195 and 1602 for clone 1 and 2 of scVACV ACAM2000-ACAM2000 DUP/HP, respectively.

Overall, the sequencing data corroborates the in vitro genomic analysis data and confirms that scVACV ACAM20000-WR DUP/HP and scVACV ACAM2000-ACAM2000 DUP/HP were successfully reactivated in SFV-infected cells.

### Removal of YFP Gpt Selection Marker

Following reactivation of the scVACV ACAM2000 YFP-gpt::105, the yfp/gpt selection marker in the thymidine kinase locus can be removed.

### Example 3. Human Mesenchymal Stem Cells (MSC) Loaded with a Synthetic Chimeric Poxvirus

Primary human MSCs are isolated from bone marrow of healthy donors, cultured, and characterized as described in Apel A et al. Exp Cell Res 2009; 315:498-507.

Human MSC are infected with the synthetic chimeric poxvirus, such as scHPXV, scVACV ACAM2000-WR DUP/HP or scVACV ACAM2000-ACAM2000 DUP/HP. Cells are infected in serum-free media 2 hours at 37° C. while in constant rotation to prevent cells from settling. Cells are infected at multiplicity of infection (MOI) of 2 or 4. After incubation, MSC are gently pelleted and supernatant removed. Cells are plated and allowed to incubate at 37° C. for 48 hours. The percent of infected cells is determined by flow cytometry.

The oncolytic activity of MSCs loaded with the synthetic chimeric poxvirus is determined by using a specific highly or intermediately proliferating cell line or in vivo.

### Example 4. Adipose-Derived Stem Cells (ADSCs) Loaded with a Synthetic Chimeric Poxvirus

Adipose tissue is an ideal source of mesenchymal stem cells. Human adipose tissues are obtained from healthy donors as fat biopsies in an outpatient clinic. 2 cubic cm of subcutaneous fat is excised prior to incision of the fascia. The fat tissues are minced with surgical scalpels and incubated in 0.075% collagenase type I (Worthington Biochemical, Lakewood, N.J.) for 90 min at 37° C. Digested tissue is centrifuged at 400 g for 5 min with the pellet washed in PBS, passed through a 70 m cell strainer (BD Biosciences, San Jose, Calif.), and incubated in red blood cell lysis buffer (154 mM NH4Cl, 10 mM KHCO3, 0.1 mM EDTA). Cells are grown in T-175 cm2 flasks at a concentration of 1.0-2.5×103 cells/cm2 in Advanced MEM with 5% PLTmax (Mill Creek Life Sciences, Rochester, Minn.), 100 U/ml penicillin, 100 g/ml streptomycin, and 2 mM L-glutamine (Invitrogen, Carlsbad, Calif., USA) in a 37° C. 5% CO2 incubator for 3-4 days. When cells are 60-80% confluent, they are passaged using TrypLE (Trypsin Like Enzyme, Invitrogen).

Cells are frozen in aliquots in liquid nitrogen and stored until use.

ADSCs are infected with the synthetic chimeric poxvirus, such as scHPXV, scVACV ACAM2000-WR DUP/HP or scVACV ACAM2000-ACAM2000 DUP/HP. Cells are infected in serum-free media 2 hours at 37° C. while in constant rotation to prevent cells from settling. Cells are infected at multiplicity of infection (MOI) of 2 or 4. After incubation, ADSCs are gently pelleted and supernatant removed. Cells are plated and allowed to incubate at 37° C. for 48 hours. The percent of infected cells is determined by flow cytometry.

The oncolytic activity of ADSCs loaded with the synthetic chimeric poxvirus is determined by using a specific highly or intermediately proliferating cell line or in vivo.

1. An isolated stem cell or population thereof comprising a synthetic chimeric poxvirus (scPV), wherein the virus is replicated and reactivated from DNA derived from synthetic DNA, the viral genome of said virus differing from a wild type genome of said virus in that it is characterized by one or more modifications.

2. The isolated stem cell or population thereof according to claim 1, wherein the poxvirus is an orthopoxvirus.

3. The isolated stem cell or population thereof according to claim 2, wherein the orthopoxvirus is selected from: camelpox virus (CMLV), cowpox virus (CPXV), ectromelia virus (ECTV), horsepox virus (HPXV), monkeypox virus (MPXV), rabbitpox virus (RPXV), raccoonpox virus, skunkpox virus, Taterapox virus, Uasin Gishu disease virus, vaccinia virus (VACV), variola virus (VARV) or volepox virus (VPV).

4. The isolated stem cell or population thereof according to claim 3, wherein the vaccinia virus strain is selected from: Western Reserve, Clone 3, Tian Tian, Tian Tian clone TP5, Tian Tian clone TP3, NYCBH, NYCBH clone Acambis 2000, Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), HD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Chorioallantois Vaccinia virus Ankara (CVA), Modified vaccinia Ankara (MVA), and MVA-BN.

5. The isolated stem cell or population thereof according to any of claims 1 to 4 that is a non-cancer stem cell.

6. The isolated stem cell or population thereof according to any of claims 1 to 5 that is a human cell.

7. The isolated stem cell or population thereof according to any of claims 1 to 6 that is selected from a mesenchymal stem cell (MSC), a neuronal stem cell, a vascular stem cell, an epidermal stem cell or an induced pluripotent stem cell.

8. The isolated stem cell or population thereof according to any of claims 1 to 7, wherein the MSC is derived from bone marrow, umbilical cord blood, or adipose tissue.

9. The isolated stem cell or population thereof according to any of claims 1 to 8 that is selected from autologous or allogeneic cells.

10. The isolated stem cell or population thereof according to any of claims 1 to 9, wherein the one or more modifications comprise one or more deletions, insertions, substitutions, or a combination thereof.

11. The isolated stem cell or population thereof according to claim 10, wherein the one or more modifications comprise one or more modifications to introduce or delete one or more unique restriction sites.

12. The isolated stem cell or population thereof according to any of claims 1 to 11, wherein the viral genome comprises heterologous terminal hairpin loops.

13. The isolated stem cell or population thereof according to any of claims 1 to 12, wherein the viral genome comprises terminal hairpin loops derived from vaccinia virus (VACV).

14. The isolated stem cell or population thereof according to any of claims 1 to 11, wherein the viral genome of the scPV comprises homologous or heterologous terminal hairpin loops and wherein the tandem repeat regions comprise a different number of repeats than the wtPV.

15. The isolated stem cell or population thereof according to any of claims 1 to 14, wherein the left and right terminal hairpin loops a) comprise the slow form and the fast form of the vaccinia virus terminal hairpin loop, respectively, b) comprise the fast form and the slow form of the vaccinia virus terminal hairpin loop, respectively, c) both comprise the slow form of the vaccinia virus terminal hairpin loop, or d) both comprise the fast form of the vaccinia virus terminal loop.

16. The isolated stem cell or population thereof according to any of claims 1 to 15, wherein the virus is replicated and reactivated from overlapping chemically synthesized DNA fragments that correspond to substantially all of the viral genome of the scPV.

17. The isolated stem cell or population thereof according to any of claims 1 to 15, wherein the virus is reactivated using a leporipox virus-catalyzed recombination and reactivation.

18. A pharmaceutical composition comprising the isolated stem cell or population thereof of any one of claims 1 to 17, and a pharmaceutically acceptable carrier.

19. The pharmaceutical composition according to claim 18, wherein the scPV is inactivated.

20. The pharmaceutical composition according to claim 19, wherein the inactivation is performed by heat, UV or formalin.

21. A method for delivering a scPV to a subject, comprising infecting the stem cells or population thereof of any of claims 1-17 with said scPV and administering the scPV-infected stem cells into the subject.

22. A method of treating or preventing cancer in a subject, comprising administering the stem cells or population thereof of any of claims 1 to 17 or the pharmaceutical composition of any one of claims 18-20 to the subject, to thereby contact the cancer cells of the subject with the scPV.

23. The method of claim 22, wherein the stem cells or population thereof of any of claims 1 to 17 or the pharmaceutical composition of any one of claims 18-20 are administered in a single administration or multiple administrations.

24. The method of claim 22, wherein the stem cells or population thereof of any of claims 1 to 17 or the pharmaceutical composition of any one of claims 18-20 are administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, ocularly, vaginally, intracoronary, intramyocardially, transendocardially, trans-epicardially, intraspinally, intra-striatumly, transdermally, rectally or sub-epidermally.

25. The method of claim 21 or 22, wherein the virus encodes a therapeutic gene product.

26. The method of claim 25, wherein the therapeutic gene product is an anti-cancer agent or an anti-angiogenic agent.

27. The method of claim 25, wherein the therapeutic gene product is selected from: a cytokine, a chemokine, an immunomodulatory molecule, an antigen, an antibody or fragment thereof, an antisense RNA, a prodrug converting enzyme, an siRNA, an angiogenesis inhibitor, a toxin, an antitumor oligopeptide, a mitosis inhibitor protein, an antimitotic oligopeptide, an anti-cancer polypeptide antibiotic, a transporter protein, or a tissue factor.

28. The method of claim 21 or 22, wherein the scPV contains a gene deletion.

29. The method of claim 28, wherein the deleted gene is selected from a gene encoding a protein or fragment thereof, a gene segment that regulates transcription, a gene segment that regulates viral replication, a gene segment that affects cellular mitosis, a gene segment that affects cellular metabolism, a gene segment that encodes an antisense RNA, a gene segment that encodes an siRNA, a gene segment that regulates angiogenesis, a gene segment that regulates one or more transporter proteins, or a gene segment that regulates one or more tissue factors.

30. The method of claim 28, wherein the gene deletion potentiates the anti-cancer or the anti-angiogenic effect of the virus.

31. A method of treating a variola virus infection, comprising administering to a subject in need thereof the stem cells or population thereof of any of claims 1 to 17 or the pharmaceutical composition of any of claims 18-20.

32. The method of claim 31, wherein the stem cells or population thereof of any of claims 1 to 17 or the pharmaceutical composition of any of claims 18-20 are administered in a single administration or multiple administrations.

33. The method of claim 31, wherein the stem cells or population thereof of any of claims 1 to 17 or the pharmaceutical composition of any one of claims 18-20 are administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, ocularly, vaginally, intracoronary, intramyocardially, transendocardially, trans-epicardially, intraspinally, intra-striatumly, transdermally, rectally or sub-epidermally.
